(19) United States
(12) Patent Application Publication
(10) Pub. No.: US 2013/0184323 A1
(43) Pub. Date: Jul. 18, 2013

(54) TREATMENT OF ENDOPLASMIC RETICULUM STRESS-RELATED DISEASES AND CONDITIONS

(71) Applicants: Doris M. Benbrook, Oklahoma City, OK (US); Franklin A. Hays, Oklahoma City, OK (US)

(72) Inventors: Doris M. Benbrook, Oklahoma City, OK (US); Franklin A. Hays, Oklahoma City, OK (US)

(73) Assignee: THE BOARD OF REGENTS OF THE UNIVERSITY OF OKLAHOMA, NORMAN, OK (US)

(21) Appl. No.: 13/779,136

(22) Filed: Feb. 27, 2013

Related U.S. Application Data
(63) Continuation-in-part of application No. 12/608,468, filed on Oct. 29, 2009, now abandoned.
Application No. 12/608,468 is a continuation of application No. 11/404,701, filed on Apr. 14, 2006, now Pat. No. 7,612,107.
(60) Provisional application No. 61/603,694, filed on Feb. 27, 2012.
(60) Provisional application No. 60/671,692, filed on Apr. 15, 2005.

(51) Int. Cl. A61K 31/382

(52) U.S. Cl. CPC A61K 31/382
USPC 514/432

(57) ABSTRACT

A method of treating a disease or condition associated with at least one of a cellular accumulation of unfolded and/or misfolded proteins, an abnormal unfolded protein response (UPR), an endoplasmic reticulum (ER) stress, and an abnormal autophagy response, is disclosed. The method may include administering one or more flexible heteroarotinoid compounds which target, bind to, disrupt, and/ or modulate the activity of one or more of heat shock proteins

### CROSS REFERENCE TO RELATED APPLICATIONS/INCORPORATION BY REFERENCE STATEMENT

The present application claims the benefit of U.S. Provisional Application No. 61/603,694, filed Feb. 27, 2012. The present application is also a continuation-in-part of U.S. Ser. No. 12/608,468, filed Oct. 29, 2009, now abandoned; which is a continuation of U.S. Ser. No. 11/404,701, filed Apr. 16, 2006, now U.S. Pat. No. 7,612,107, issued Nov. 3, 2009; which claims benefit under 35 U.S.C. 119(e) of U.S. Ser. No. 60/671,692, filed Apr. 15, 2005. The entire contents of each of the above-referenced patents and patent applications are hereby expressly incorporated herein by reference.

### STATEMENT REGARDING FEDERALLY SPONSORED OR DEVELOPMENT

This invention was made with government support under Contract Number CA106713 awarded by the National Institutes of Health (NIH). The government has certain rights in the invention.

### BACKGROUND

Protein folding diseases may occur when specific proteins remain unfolded or are misfolded after their synthesis, leaving them unable to perform their particular function. A protein is first made inside the endoplasmic reticulum (ER) of the cytoplasm as a chain of linked amino acids that must be folded in a certain way in order for the protein to be able to perform its specific function. Only correctly folded proteins can be transported to the Golgi apparatus. Unfolded or misfolded proteins can cause harm to the cell due to the loss of the appropriate protein function and also by forming aggregates that can disrupt the protein synthesis and degradation machinery (24) and sequester transcription factors (25, 26). The exposed hydrophobic amino acids in the unfolded or misfolded forms drive both protein aggregation and binding to certain heat shock proteins (HSPs).

Accumulation of unfolded proteins in the cell leads to ER stress (ERS) in the cell and initiates the unfolded protein response (UPR). The UPR protects cells from the toxic effects of accumulation of unfolded proteins by slowing down protein synthesis, increasing transcription of ER chaperones that bind to unfolded/misfolded proteins and guide their appropriate folding, and by increasing transcription of genes that promote ER associated degradation (ERAD). ERAD is a process that uses HSPs to unfold and properly refold misfolded proteins and also sends the unfolded proteins back to the cytosol for immediate degradation in the proteasome. If these functions are not successful in preventing serious injury to the cell caused by the accumulation of unfolded/misfolded proteins, then UPR initiates cell death through apoptosis pathways in order to eliminate the diseased cells and ensure survival of the organism.

Heat shock proteins have been considered to be viable targets for drug development (1-3). Drugs that inhibit proteasomal degradation induce ERS by increasing the unfolded/misfolded protein-to-chaperone ratio and have demonstrated clinical efficacy. One of these, Bortezomib (VELCADE®, Millenium Pharmaceuticals, Inc., Cambridge, Mass.) is approved by the USFDA for treatment of refractory multiple myeloma (4). It has become increasingly clear in cancer therapy that targeting single molecules is not as effective as simultaneously targeting several molecules and there are ongoing cancer clinical trials with agents targeted at the related HSP90 protein in combination with Bortezomib (5).

As indicated above, several HSPs (including, but not limited to HSPA5, HSPA8, and HSPA9) are considered to be important protein chaperones. HSPA5 (also called BiP or GRP78) is the key sensor for imbalance between unfolded/misfolded proteins and chaperones. When the number of unfolded or misfolded proteins exceeds the number of chaperones available to fold them, HSPA5 is attracted to the excess of unfolded/misfolded proteins. HSPA5 binds to unfolded proteins through a substrate binding domain and guides their folding by undergoing a conformational change driven by hydrolysis of ATP in an ATPase domain. Co-chaperones belonging to the HSP40 family regulate the ATPase activity of HSPA5, namely ER dnaJ(ERdj)1, Erdj3/human ER-associated dnaJ (HEDJ), ERdj4, ERdj5, SEC63 and P58IPK. Exchange of ADP for ATP is facilitated by BiP-associated protein (BAP), a member of the GrpE family. During ERAD, HSPA5 binds nascent proteins and partially unfolds them by cleaving their disulfide bonds.

During cell homeostasis, HSPA5 is bound to the ER lumen side of three proteins, PERK (eIF2alpha kinase), and ATF6 (activating transcription factor 6) and IRE1 (inositol requirement 1) that are ER transmembrane proteins and mediators of ERS. Upon accumulation of unfolded/misfolded proteins, HSPA5 leaves these three proteins to bind the unfolded/misfolded proteins. Release of HSPA5 from binding to these three ERS mediator proteins enables them to perform their various functions in mediating the cell response to ERS including reducing new protein synthesis, increasing the number of chaperones available for protein folding, and increasing ERAD to eliminate the excessive load of proteins needed to be folded properly. Upon release from HSPA5, PERK is free to phosphorylate elF2alpha which causes translational attenuation of most proteins, but certain proteins, such as ATF4 and nuclear factor kappa B (NF-κB) are induced. Induction of ATF4 leads to induction of the transcription factor CHOP, a key factor in mediating apoptosis in response to ER stress. CHOP down-regulates expression of the anti-apoptotic factor BcI-2 (9) and induces expression of the pro-apoptotic factor Tribbles homolog 3 (TRB3) (27). PERK also contributes to induction of apoptosis by phosphorylating nuclear respiratory factor (NRF)2. Upon release from HSPA5, ATF6 is transported to the Golgi apparatus where it is cleaved into an active transcription factor that also induces transcription of CHOP as well as HSPA5. Release of IRE1 from HSPA5 allows IRE1 to alternatively splice x-box binding protein1 (XBP1) mRNA resulting in a transcription factor with alternate target genes involved in ERAD, lipid synthesis, ER biogenesis and ER chaperones including HSPA5. IRE1 is also involved in the induction of ER-stress induced apoptosis by forming a complex with TRAF2 and ASK1, which leads to phosphorylation activation of Jun N Kinase (JNK) (14).

ERS can induce autophagy directly through upregulation of HSPA5 and through mechanisms downstream of HSPA5 release of the three UPR signal transducers. A critical role for HSPA5 in the induction of autophagy was demonstrated with knockdown of HSPA5 in normal and cancer cells, which prevented autophagosome formation in response to starvation or in response to inhibition of protein processing with tunicamycin, an inhibitor of N-linked glycosylation (10). This study also provided evidence for an integral co-dependency of intact ER and autophagy. The massively dilated and disrupted ER and the deficient autophagosome formation induced by HSPA5 knockdown were both alleviated by simultaneous knockdown of the XBP-1 transcription factor, a downstream UPR mediator of IRE1a action required for ER expansion (12), suggesting that intact ER is maintained by and/or required for autophagy. The link between HSPA5 and autophagy induction occurs downstream of nucleation, as the HSPA5 knockdown had no effect on Beclin 1/Vps34 association.

Diabetes is a condition and disease which is associated with ERS. Protein biosynthesis (e.g., of insulin) that overwhelms the ER protein folding machinery to the extent that an accumulation of misfolded/unfolded proteins occurs in the cell results in ERS. This activates the adaptive UPR pathway, which upregulates production of protein-folding chaperones, attenuates protein translation, and initiates ER-associated protein degradation. Type 2 Diabetes Mellitis (T2DM) is a prevalent metabolic disease often related to obesity and is associated with insulin resistance in peripheral tissues, hyperglycemia, hyperinsulinemia, and eventual loss of the cells (β-cells of the Islets of Langerhans) which produce insulin. As blood glucose levels increase in an individual who has T2DM, there is a compensatory increase in insulin production by the pancreatic β-cells to prevent hyperglycemia. This increased insulin secretion from pancreatic (β-cells drives insulin signaling in peripheral tissues to activate intracellular anabolic pathways resulting in euglycemia. Increased insulin production in the β-cells and increased protein synthesis in insulin-sensitive cells is intimately tied to ER homeostasis since the ER is required for proper chaperone-assisted protein folding. However, as increased demands for protein folding surpass protein folding capacity in the ER, there is an increase in unfolded or misfolded proteins in the ER lumen, causing ERS (59). For example, amylin, also known as islet amyloid polypeptide (IAPP), is secreted from pancreatic islet β-cells as a physiological component of insulin granules. Amylin forms aggregrates that are found accumulated in pancreatic islets in 40-60% of autopsies performed on individuals who had T2DM. Because pancreatic islet amyloid is not found in autopsies of persons who did not have disturbances in glucose metabolism, this is considered a pathological characteristic of T2DM. Pancreatic islet amyloid induces ERS and upregulation of HSPA5, and can cause diabetes in animals when artificially overexpressed in their pancreatic islet cells.

As indicated, ERS activates the UPR pathway which functions to (1) upregulate protein folding chaperones, such as the 78 kDa folding chaperone HSPA5 (a.k.a., glucose regulated protein 78, or GRP78) which mitigate accumulation of unfolded proteins; (2) limit protein translation; (3) initiate ER-associated protein degradation (ERAD) to decrease unfolded protein concentration; and (4) ultimately initiate apoptosis if ER homeostasis is not restored. Steps 1-3 of the UPR are referred to as “adaptive” UPR since they result in decreasing ERS and preserving cellular homeostasis. However, step 4 (initiation of cell death by apoptosis) occurs when the adaptive UPR is not capable of reducing tonic ERS in the cell and is driven, in part, by the protein phosphatase 1 regulatory subunit 15A protein (also called Growth arrest and DNA damage-inducible protein (GADD34)) and C/EBP homologous protein (CHOP). Failure of the UPR to normalize ERS leads to irreversible damage and apoptosis that, in T2DM, results in a reduction of β-cell mass, insulin resistance, and tissue-specific pathology in peripheral tissues (53). Thus, adaptive UPR is beneficial to cell survival in the presence of ERS as it promotes production of protein folding chaperones, decreases protein production, and increases protein degradation. This primes the cell to respond to future ERS and, as a result, facilitates long-term protein production (e.g., insulin production) in high stress environments while preventing cell death. There is emerging clear evidence that adaptive UPR activation has beneficial effects in pancreatic β-cells (60) and in T2DM (53-55, 61). HSPA9 (mortalin) also plays a role in diabetes. The post-translational modification of the H5PA9 protein was found to be altered in an animal model of diabetes (53). The HSPA9 protein was identified in 2D-gel analysis as being up-regulated by cytokines beta-cell line (RIN-cells) and in rat islets of Langerhans (54). Higher IL-1 beta-induced HSPA9 expression was observed in islets from rats with increased susceptibility to diabetes. Constitutive over-expression of HSPA9 in cells can decrease cell survival (55). Taken together these results support a role of HSPA9 in mediating cytokine-induced death of pancreatic beta cells resulting in reduced insulin production and diabetes.

As noted above, if adaptive UPR is unable to restore ER homeostasis then pro-apoptotic pathways are activated leading to cell death. In diabetes, the adaptive UPR is linked to preservation of insulin secreting β-cells and peripheral insulin signaling (53-55). Thus, therapeutics aimed at decreasing ERS and promoting adaptive UPR would be of significant benefit in the clinical treatment of diseases and conditions involving ERS such as T2DM and also in preserving the “honeymoon phase” in Type 1 diabetes (T1DM), which is a period of time during T1DM before autoimmunity entirely destroys the β-cells of the pancreas. As noted above, HSPA5 functions as a master regulator of UPR in response to ERS (54) and is recognized as a viable drug target for the treatment of diabetes (53, 56). HSPA5-dependent activation of adaptive UPR can decrease ERS in peripheral tissues and β-cells. This decrease will reduce insulin resistance and preserve hypersecretory β-cells and thus β-cell mass in obesity-induced T2DM, for example.

Many diseases are associated with the loss of cell survival processes that can arrest cell metabolism and proliferation, and eliminate damaged molecules. If the damage can be repaired, these processes will re-establish cellular activity. If the damage cannot be sufficiently repaired, these processes shunt the cell into a programmed cell death pathway in order to prevent damaged cells from accumulating and causing disease. As noted above, HSPs detect and correct unfolded or misfolded proteins and initiate cell survival responses. They also stabilize proteins and protein complexes in properly folded conformations, thereby allowing their function and preventing accumulation of unfolded or misfolded proteins or malformed aggregates associated with numerous diseases. The HSPs are integral components of cell function that not only bind to unfolded proteins in order to assist in their proper folding, but also modulate assembly of multi-protein complexes. Some of the HSP clients are key regulatory proteins which control cell survival responses, including cyclin D1, BcI-2 and p53. Numerous diseases, including cancer, are associated with overexpression of these key regulatory proteins to the extent that the diseased cells are addicted to them and cannot survive without them.

Malfunction of HSPs is therefore a key characteristic of numerous diseases that causes the diseased cells to accumulate unfolded/misfolded or damaged proteins, or to have defective survival responses in comparison to corresponding healthy cells. As noted, HSPs can function differently in stressed cells in comparison to normal cells, by driving the key survival proteins to shunt cells away from the alternative programmed cell death pathway in diseased cells only and confer a survival advantage (23a). Thus, inhibiting certain HSP interactions will exert a selective effect on diseased cells while leaving healthy cells unaffected. In some diseases or conditions, HSP overexpression or malfunction allows the cells to bypass regulatory controls over cell proliferation and programmed cell death, which allows the cells to survive even with significant damage. Among these diseases and conditions are: cancers, polycystic kidney disease (PKD) and viral infection.

Individuals with a family history of cancer often harbor mutations in DNA damage response and repair genes, such as BRCA1. Because BRCA1 overexpression stabilizes wild-type p53 and directs its effect toward the survival pathway, loss of BRCA1 in these genetically predisposed patients puts their cells at increased risk for being shunted into the p53-driven programmed cell death pathway and more dependent upon HSPA9 prevention of p53-induced apoptosis (23a).

Another cellular mechanism which eliminates unfolded/misfolded and damaged proteins is autophagy. There are three natural process of autophagy in the cell, macroautophagy, microautophagy and chaperone-mediated autophagy. Macroautophagy is a natural process in which portions of the cell, including organelles, are engulfed in double-membraned vesicles called autophagosomes. The autophagosomes eventually fuse with lysosomes to form autophagolysosomes where the internal molecules are digested and their components released for recycling within the cells. Chaperone-mediated autophagy (CMA) is driven by HSPA8 (8). Mucolipidosis Type IV (MLIV) has been shown to have defective CMA (9). MLIV is caused by mutations in the MCOLN1 gene, which encodes the transient receptor potential mucolipin-1 (TRPMLI) protein that is localized to lysosomes. MLIV defects in the TrPMLI gene are speculated to cause the disease by ineffective docking with HSPA8 inside the lysosome (9). In microautophagy, defective molecules or organelles are directly engulfed into the lysosomes for degradation and recycling of their components.

HSPA8 (also called Hsc70 or HSP73) is located in the cytoplasm, expressed in all cell types, and considered to be a housekeeping member of the family (28). It is an essential cofactor of the ubiquitin machinery needed for proteasomal degradation (29), controls intracellular trafficking of proteins (30), and enhances intracellular uptake and degradation of proteins by facilitating lysozomal degradation of proteins (31). HSPA8 binds and stabilizes newly synthesized cyclin D1 thereby increasing its availability for assembly into a complex with cyclin dependent kinase 4 (CDK4), which functions to promote progression of cells from the G1 resting phase into the S phase of DNA replication (16). Knockdown of HSPA8 with siRNA disrupted the ability of endothelial cells to form tubes (18). Binding of HSPA8 with a small molecule inhibitor caused HSPA8 client protein degradation and induced cell cycle arrest and apoptosis (6), while over-expression of HSPA8 can protect myocardial cells in vitro and in vivo (32-35). After ischemia reperfusion injury, heat stress induces HSPA8 in correlation with protection of ventricular and endothelial function (36, 37). Deletion of HSPA8 causes dysfunctional cardiomyocytes and incomplete stress response of heart upon ischemia/reperfusion injury (3S) HSPA8 is required for transport of proteins along neuronal axons, which is implicated in the development of glaucoma and other neuronal transport diseases (39). HSPA8 is the only chaperone which causes chaperone-mediated autophagy by binding to proteins that expose a specific motif and bringing them directly to the lysosome where they are transported through the LAMP2 receptor to a lysosomal HSPA8 waiting inside the lysosome (52).

HSPA9 (also known as mortalin) also contains a substrate binding domain and an ATPase domain. Mutations in HSPA9 causing mitochondrial dysfunction have been demonstrated in patients with Parkinson's Disease (40). Altered cellular distribution of HSPA9 from a homogenous staining throughout the cytoplasm to a peri-nuclear localization was consistently observed in normal versus cancer cells, respectively, and was reversed upon scenescence (41). Although HSPA9 can be found in the ER, cytoplasmic vesicles and cytosol (42, 43), the majority of the protein present in the cell is located within mitochondria (44, 45). HSPA9 plays a crucial role in import of proteins into the mitochondria through translocases in both the outer and inner membranes by providing the necessary ATPase component and facilitating the unfolding and refolding of the protein required for import (22, 23). It also interacts with p66Shc to maintain mitochondrial membrane potential (23). HSPA9 binds to and inhibits the ability of p53 to induce apoptosis in stressed cells, but not in weakly stressed or unstressed cells (24).

TABLE 1
Molecular, cellular organelle and cellular physiological events elicited by HSPA5,
HSPA8 and HSPA9 that are regulated by HSPA5, HSPA8, and HSPA9-binding

| Activity | HSPA5/8/9 effect | HSPA5/8/9 Binder effect |
|---|---|---|
| Cellular homeostasis | Restoration of cellular homeostasis if damage | Reversal of the diseased phenotype in a subset |
| and apoptosis | can be repaired, but induction of apoptosis in | of the diseased cell population and induction of |
| | the event of irreparable damage (1-3). | apoptosis in another subset of the diseased |
| | | population (7) |
| ER stress | HSPA5 is the primary sensor of ER stress and | ER stress observed by electron microscopy and |
| | mediator of the UPR leading to upregulation (1-3). | expression of ERstress protein markers, IRE 1α, |
| | | PDI, Ero I-Lα, HSPA5 and CHOP are induced by |
| | | treatment (8). |
| Bcl-2 anti-apoptotic | Release of HSPA5 binding to PERK | Decreased levels of Bcl-2 in association with |
| protein expression | causes phosphorylation of eIF2α causing | apoptosis in cancer cells, but not in healthy cells |
| | upregulation of ATF4, which induces expression | (10, 11). |
| | of CHOP, which inhibits expression of Bcl-2 (9). | |
| Autophagy | HSPA5 is required for autophagy (10) | Induction of autophagy (11) |
| NE-κB activity | HSPA5 release of PERK induces NF-κB activity | Inhibition of NF-κB activation |
| | through induction of IKK (12). | through indirect repression of IKK |
| | | (13). |
| JNK phosphorylation | Release of HSPA5 binding to IRE1α allows it to | Induction of JNK phosphorylation |
| | form a complex with TRAF2/ASKI, which | (15). |
| | phosphorylates JNK(14). | |
| Cyclin D1 levels | HSPA8 binds and stabilizes newly | Flex-Hets prevent G1 to S phase |
| | synthesized cyclin D1 thereby increasing its | progression by decreasing the levels of cyclin D1 |
| | availability for assembly into a complex with | and the down-stream signaling induced by the |
| | cyc1in dependent kinase 4 (CDK4), which | cyclin D1/CDK4 complex (17). |
| | functions to promote progression of cells from | |
| | G1 resting phase into S phase of DNA | |
| | replication (16). | |
| Endothelial tube | HSPA8 is required for endothelial | SHetA2 inhibits endothelial tube |
| formation | tube formation (18) | formation (19). |
| Mitochondrial | HSPA9 is required for Mitochondrial Complex I | Inhibition of Mitochondrial Complex I activity |
| complex 1 activity | activity (20). | (21), |
| Mitochondrial | HSPA9 is required for import of proteins | Induction of mitochondrial swelling within 15 |
| translocases | through mitochondrial translocases (22, 23) | minutes of treating cancer cells, with no effect |
| | | on mitochondria in normal cells (10). |
| p66Shc | Binding by HSPA9 prevents loss of | Loss of mitochondrial membrane |
| | mitochondrial trans-membrane | potential within 15 minutes of treating cancer |
| | potential (23). | cells, with no effect on mitochondria in normal |
| | | cells (10). |
| p53 | HSPA9 binds to and inhibits the ability of p53 to | Induction of ER stress and apoptosis in cancer |
| | induce apoptosis in stressed cells, but not in | cells, but neither in normal cells (7, 10, 11). |
| | weakly stressed or unstressed cells (23a). | |

From Zhao, L., et al. (3)

TABLE 2
Alternate names/Synonyms of HSPA5/8/9

| Heat | |
|---|---|
| Shock | |
| Protein | Synonyms (Bolded names are most commonly used) |
| HSPA5 | BiP, GRP78, 78 kDa, AL022860, AU019543, BIP, |
| | D2Wsu141e, D2Wsu17e, FLJ26106, HEAT SHOCK |
| | 70 KDA PROTEIN5, Hsce70, HSP70-5, Immunoglobulin |
| | heavy chain binding, mBiP, MIF2, SEZ-7 |
| HSPA8 | Hsc70, 2410008N15Rik, HEAT SHOCK COGNATE 71-KDA, |
| | HEAT SHOCK PROTEIN 8, HS7C, HSC54, HSC71, HSC73, |
| | HSP/C70, HSP70, HSP71, HSP73, Hsp 8, HSPA10, |
| | LAP1, MGC102007, MGC106514, MGC114311, |
| | MGC118485, MGC128130, MGC131511, |
| | MGC29929, NIP71 |
| HSPA9 | Mortalin, GRP-75, 74 kDa, CSA, Hsc74, Hsp74, Hsp74a, |
| | Hspa9a, HSPA9B, MGC4500, Mitochondrial stress-70, |
| | MOT, Mot1, MOT2, Mthsp70, MTHSP75, PBP74, Stress-70 |

Various molecular, cellular organelle, and cellular physiological events elicited by HSPA5, HSPA8, and HSPA9 that are regulated by binding of HSPA5, HSA8, and HSPA9 are shown in Table 1. Synonyms for these HSP proteins are listed in Table 2.

### BRIEF DESCRIPTION OF THE DRAWINGS

FIG. 1 depicts SDS gels of wash and eluent from magnetic beads. Lane 1: MW markers; Lane 2: empty; Lane 3: First wash from empty beads; Lane 4: First wash from SHetA2 beads; Lane 5: Second wash from empty beads; Lane 6: Second wash from SHetA2 beads; Lane 7: Eluent from empty beads using excess SHetA2; Lane 8: Eluent from SHetA2 beads using excess SHetA2. Arrow indicates 75Kd band which was used for mass spec analysis. This experiment was reproduced 3 times.

FIG. 2 demonstrates SHetA2 inhibition of colorectal cancer. There were 10 mice per treatment group. Six week old mice were treated with control diet for 3 days prior to initiation of the diet containing SHetA2 compound or control vehicle. Feeding was continued for 13 weeks at which time the mice were necropsied and their tumors counted, measured and harvested for histopathology and biochemical analysis. A. Effect of SHetA2 on tumor size. B. Effect of SHetA2 on tumor incidence.

FIG. 3 is a biomarker analysis in colorectal tumors treated with SHetA2. A. Immunohistochemical analysis reveals inhibition of proliferation markers, including cyclin D1, in treated tumors. B. Western blot analysis of pooled protein extracts from 3 tumors of each treatment group demonstrates SHetA2 alteration of molecules previously shown to be similarly altered in ovarian cancer.

FIG. 4 demonstrates the functional roles of HSPA8 and HSPA9 in protection of healthy. cells from SHetA2 toxicity. Normal human ovarian surface epithelium (HOSE) cells from a woman who has not family history of ovarian cancer (A), HOSE from a woman who has a family history of/is genetically predisposed to ovarian cancer (FH-IOSE) or a human ovarian cancer cell line (A2780) were transfected separately with negative control siRNA (Neg Ctrl), or siRNA that reduces levels of HSPA8 or HSPA9 using PepMute™ Reagent (SignaGen® Laboratories, Rockville, Md.). After 24 hours, cells were plated into 96 well plates at equal cell densities. After 72 hours of treatment, the MTS cytotoxicity assay was performed. The Optical Density (OD) was measured as representative of the numbers of cells present in each well. The growth index was derived by dividing the OD of the treated cultures by the average OD of the untreated control cultures exposed to the same volume of solvent as used in the treatment.

FIG. 5 demonstrates that SHetA2 at high doses induces HSPA5 expression in diseased cells, but not in healthy cells. A. Western blots of protein extracts isolated from the human ovarian cancer cell lines (SK-OV-3 and A2780) and primary cultures of human ovarian surface epithelium (HOSE) treated with 10 μM SHetA2 for the indicated period of time were probed with antibodies to HSPA5 or a loading control housekeeping protein (βactin or GAPDH). B. The indicated cell lines were treated with the indicated concentrations of SHetA2 for 72 hours in the absence (SHetA2) or presence of salubrinol (A2+Sal) to inhibit the “adaptive response” or Thapsigargin (A2+Tp) to induce ER stress in triplicate. The growth index was determined with the MTS cytotoxicity assay by dividing the optical density (OD) output of the assay for the treated cultures by the average (OD) of control cultures treated with solvent (dimethyl sulfoxide) only.

FIG. 6 depicts electron micrographs (magnification 1800× or 7000×) of human ovarian cancer cells treated with SHetA2 which demonstrate the induction of autophagosomes, autophagolysonnes and reduction of cell size. Image A: Control cells were treated solvent only. Image B: treated cells were exposed to 10 μM SHetA2. Incubation time was 17 hrs. The photos were taken at the same magnification (1800×) showing the smaller sizes of the treated cells. Autophagosomes appear as shaded circles, cleared auotophagolysomes appear as clear circles and lysosomes appear as dark circles. Image C is an enlarged porition of Image B magnified at 7000×.

FIG. 7 demonstrates induction of acidic vesicles in diseased cells treated with SHetA2. Ovarian cancer cells (upper panel) and normal healthy HOSE cells (lower panel) were treated with SHetA2 for the indicated amount of time before staining with acridine orange and visualization with fluorescent microscopy.

FIG. 8 is a Western blot which demonstrates time course of LC3-II induction by SHetA2 in diseased cancer cells. Equal amounts of protein extracts isolated from ovarian cancer cells treated with 10 μM SHetA2 for the indicated amount of time were electrophoresed into an SDS gel and blotted onto a membrane. Western blots of the membrane were performed using an antibody that recognizes both forms I and II of LC3. The blots were stripped and reprobed with an antibody to GAPDH to measure as a control for equal protein loading in each lane.

FIG. 9 demonstrates that SHetA2 provided at 10 μM induces ER stress and HSPA5 (BiP) expression in ovarian cancer cells. (A). Electron microscopic images demonstrating SHetA2 induction of ER stress in SK-OV-3 diseased human ovarian cancer cells. SK-OV-3 cells grown in absence (solvent control) or presence of 10 μM SHetA2 for 17 hrs. M: Mitochondria. White arrows:swollen mitochondria. Black oval:non-swollen ER. Black arrows: swollen ER surrounded by ribosomes. Lines at bottom: 500 nm. Each picture was taken at 120,000× from a single experiment and representative of 3 independent experiments. (B). Western blots of equal amounts of protein extracted from SK-OV-3 diseased human ovarian cancer cell line cultures treated with 10 μM SHetA2 (numbers only) or solvent only (24c) for the indicated time, and probed with a monoclonal antibody specific for BiP or βactin as a loading control.

FIG. 10 demonstrates that SHetA2 provided at 10 μM interferes with HSPA9 interaction with its substrate proteins, p66Shc and p53. Human breast cancer cells (MDA-MB-231 cell line) were treated with 10 μM SHetA2 or the same volume of DMS0 solvent (control). After 1 hour of treatment, protein extracts of the cells were prepared and incubated with an antibody to HSPA9. After rinsing away non-specific binding proteins, the proteins bound to the antibody were immunoblotted and the Western blot was probed with antibodies to p66 Shc and p53. Reduced levels of p66 Shc and p53 were observed in the SHetA2 treated cultures.

FIG. 11 demonstrates the effect of a low dose (1 μM) of SHetA2 on biomarker proteins of UPR in response to ER stress. Approximately 1 million rat liver cells (FAO)/ well were plated in 6-well plates and grown 24 hours at which point fresh DMSO solubilized SHetA2 (1 μM) was added to each well. Cells were harvested at the indicated timepoints (2-24 hours), spun down, lysed via solubilization with M-PER, and total protein concentration in each lysate was determined by BCA. Normalization was determined by GAPDH load per lane. The lose dose of SHetA2 induced expression of biomarkers characteristic of the UPR, including HSPA5, GRP94, GADD34 and XBP-1.

FIG. 12 demonstrates the effect of increasing SHetA2 on growth of three cell lines. FAO (liver cells), HIT-T15 (beta cells), and 3T3-L1 (differentiated adipocytes) were plated in 6-well plates and grown for 24 hours followed by treatment with fresh DMSO solubilized SHetA2 at the stated concentrations. Cells were grown for 48 hours after treatment and viability was quantified using an MTT assay measured at 570 nm. Statistical error was determined from 3 independent experiments. SHetA2 is not cytotoxic to FAO liver cells, differentiated 3T3-L1 adipocytes, and HIT-T15 8-cells at low levels (up to 5 μM). Toxicity to the liver cells and beta cells increases at higher levels (10 μM).

### DETAILED DESCRIPTION

The presently disclosed inventive concept(s) is directed to treatment of diseases or conditions involving defective protein folding, in particular those associated with ERS, by administering one or more flexible heteroarotinoids (“Flex-Hets”) as described herein to a subject having such disease or condition.

Before further explaining at least one embodiment of the presently disclosed inventive concept(s) in more detail by way of exemplary description, examples, and results, it is to be understood that the presently disclosed inventive concept(s) is not limited in its application to the details of methods and compositions as set forth in the following description. The presently disclosed inventive concept(s) is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concept(s) may be practiced without these specific details. In other instances features which are well known to persons of ordinary skill in the art have not been described in detail to avoid unnecessary complication of the description.

Unless otherwise defined herein, scientific and technical terms used in connection with the presently disclosed and claimed inventive concept(s) shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and methods of their application disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the presently disclosed inventive concept(s).

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” Throughout this application, the term “about” is used to indicate that a value includes the inherent variation of error for the composition, the method used to administer the composition, or the variation that exists among the study subjects. The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or [AB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, or at least 85% of the time, or at least 90% of the time, or at least 95% of the time, or at least 98% of the time.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism without reference to how the active agent has its physiological effects.

As used herein, “pure,” or “substantially pure” means an object species is the predominant species present (i.e., on a molar basis it is more abundant than any other component in the composition thereof), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species. The term “pure” or “substantially pure” also refers to preparations where the object species is at least 60% (w/w) pure, or at least 70% (w/w) pure; or at least 75% (w/w) pure; or at least 80% (w/w) pure; or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (/w) pure, or at least 99% (w/ w) pure, or 100% (/w) pure.

“Treatment” refers to therapeutic treatments. “Prevention” refers to prophylactic or preventative treatment measures. The term “treating” refers to administering the composition to a patient or subject for therapeutic purposes. Treatment” refers to both therapeutic treatment and prophylactic or preventative measures. Those in need of treatment include, but are not limited to, individuals already having a particular condition or disease as well as individuals who are at risk of acquiring a particular condition or disease (e.g., those needing prophylactic/preventative measures). The term “treating” refers to administering an agent to a subject for therapeutic and/or prophylactic/preventative purposes.

A “therapeutic composition” or “pharmaceutical composition” refers to a composition comprising a flexible heteroarotinoid (“Flex-Het”) that may be administered topically to bring about a therapeutic effect as described elsewhere herein.

A “disease” or “condition” is any physical state of a subject that would benefit from treatment with the Flex-Hets described herein. This includes chronic and acute disorders, conditions, or diseases including those pathological conditions which predispose the mammal to the disorder or disease in question, such as a predisposition to diabetes. The terms “cancer” and “cancerous” refer to or describe the physiological condition in mammals that is typically characterized by unregulated cell growth.

The term “effective amount” refers to an amount of a Flex-Het described herein that is sufficient to exhibit a detectable therapeutic effect without undue adverse side effects (such as toxicity, irritation and allergic response) commensurate with a reasonable benefit/risk ratio when used in the manner of the inventive concept(s). The therapeutic effect may include, for example but not by way of limitation, treating a diabetic condition or inhibiting the growth of undesired tissue or malignant cells or other disease or condition described herein. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As defined herein, a therapeutically effective amount of a Flex-Het as presently disclosed more particularly refers in one embodiment to an amount which is effective in controlling, mitigating, reducing, or inhibiting a disease or condition as described herein, such as a disease or condition related to ERS. Without wishing to be bound by theory, these compounds are thought to have their actions by binding to or affecting the activity of at least one of HSPA5, HSPA8, and HSPA9 proteins. The term “controlling” is intended to refer to all processes wherein there may be a slowing, interrupting, arresting, stopping, or reversing of the progression of the disease or condition associated with ERS and does not necessarily indicate a total elimination of all disease symptoms.

As used herein, the term “concurrent therapy” is used interchangeably with the terms “combination therapy” and “adjunct therapy”, and will be understood to mean that the subject in need of treatment is treated or given another drug for the condition in conjunction with the pharmaceutical compositions of the presently disclosed inventive concept(s). This concurrent therapy can be sequential therapy, where the patient is treated first with one composition and then the other, or the two compositions are given simultaneously.

The terms “administration” and “administering”, as used herein will be understood to include administration to the subject by any suitable method which may be understood to include oral delivery of the active agent. In addition, the compositions of the presently disclosed inventive concept(s) (and/or the methods of administration of same) may be designed to provide delayed, controlled or sustained release using formulation techniques which are well known in the art, wherein for example, a Flex-Het compound continues to be released into the bloodstream for some minutes or hours after initial administration.

The presently disclosed inventive concept(s) includes a therapeutic composition comprising a therapeutically-effective or pharmaceutically-effective amount of at least one Flex-Het described herein in combination with a pharmaceutically acceptable carrier. As used herein, a “pharmaceutically acceptable carrier” is a pharmaceutically acceptable solvent, suspending agent or vehicle for delivering the compositions of the presently disclosed and claimed inventive concept(s) to the subject. The carrier may be liquid or solid and is selected with the planned manner of administration in mind. Examples of pharmaceutically acceptable carriers that may be utilized in accordance with the presently disclosed inventive concept(s) include, but are not limited to, polyethylene glycol (PEG), polymers, liposomes, ethanol, DMSO, aqueous buffers, solvents, oils, DPPC, lipids, and combinations thereof.

A “therapeutically effective amount” of a Flex-Het compound of the presently disclosed inventive concept(s) refers to an amount which is effective in controlling, reducing, or inhibiting a disease or condition described herein. The term “controlling” is intended to refer to all processes wherein there may be a slowing, interrupting, arresting, or stopping of the progression of the infection and does not necessarily indicate a total elimination of the infection symptoms.

The term “therapeutically effective amount” is further meant to define an amount resulting in the improvement of any parameters or clinical symptoms characteristic of a particular disease or condition. The actual dose will vary with the subject's overall condition, the seriousness of the symptoms, and counter indications. As used herein, the term “therapeutically effective amount” also means the total amount of each active component of the pharmaceutical composition or method that is sufficient to show a meaningful benefit. When applied to an individual active ingredient administered alone, the term refers to that ingredient alone. When applied to a combination, the term refers to combined amounts of the active ingredients that result in the therapeutic effect, whether administered in combination, serially, or simultaneously.

As used herein, the term “subject” or “patient” refers to a warm blooded animal, particularly a mammal, which is afflicted with a condition or disease described herein. It is understood that guinea pigs, dogs, cats, rats, mice, horses, goats, cattle, sheep, zoo animals, livestock, monkeys, primates, humans, and any other animals with mammary tissue are examples of animals within the scope of the meaning of the term.

As noted above, another process by which unfolded or misfolded proteins are eliminated is autophagy. In another embodiment of the presently disclosed inventive concept(s), the Flex-Hets described herein are used to stimulate the process by which autophagolysosomes are formed, fuse with lysosomes, and are cleared of their materials, and thus can be used to treat diseases in which unfolded/misfolded proteins accumulate in the cell, including unfolded protein diseases and lysosomal storage diseases. Examples of lysosomal storage diseases which can be treated with the Flex-Hets are described hereinbelow.

In many diseases, the survival responses induced by HSPA proteins are dysfunctional or bypassed to avoid cells being shunted into the alternative pathway of programmed cell death. It is considered herein that the survival responses induced by agents that bind to heat shock proteins protect normal cells, and cells associated with accumulation of unfolded/misfolded or incompletely processed proteins, which helps these cells to clear the damaged proteins and recover from cellular stresses.

The presently disclosed inventive concept(s) is also directed to inducing survival responses and clearance of damaged proteins in cells by modulating HSPA protein function. An objective of the presently disclosed inventive concept(s) is, in one embodiment, to treat diseases and conditions characterized by abnormal protein folding and ERS by upregulating the UPR and/or by eliminating severely damaged diseased cells, while preserving salvageable and normal healthy cells, by targeting and/or modulating the activity of at least one of three specific proteins involved in maintaining homeostasis of protein synthesis, folding, intracellular transport and degradation including HSPA5, HSPA8, and HSPA9.

The presently disclosed inventive concept(s) is unique in that it is directed to methods to target one, two, or all three of the heat shock proteins HSPA5, HSPA8, and HSPA9, which are located in different compartments in the cell. In one embodiment, this multipronged attack induces death in those cells that have irreparable damage and restores the normal phenotype in those cells that can be repaired.

Deregulated mitochondrial metabolism and protein homeostasis in diseased cells make such cells more susceptible to interference with heat shock protein function than non-diseased healthy cells. The presently disclosed inventive concept(s) is, in one embodiment, directed to Flex-Hets that can bind one, two, or all of HSPA5, HSPA8 and HSPA9 thereby differentially affecting cancer cells over normal cells. The flex-het drug SHetA2, for example, has undergone extensive preclinical testing at the National Cancer Institute, and no evidence of toxicity has yet been found. As shown in Table 1, all of the signaling events leading to regulation of growth and differentiation by the HSPA5/8/9 binding-Flex-Hets are known down-stream events of HSPA5/8/9 action.

Application of the Flex-Hets to certain diseased cells will cause loss of survival proteins to which these cells are addicted, which in turn will ultimately lead to their deaths. On the other hand, if the cells are not severely damaged or moderately stressed, this intervention will reverse the diseased phenotype and restore normal cellular homeostasis, if the cellular damage can be repaired or the accumulated proteins can be eliminated, for example, through autophagy.

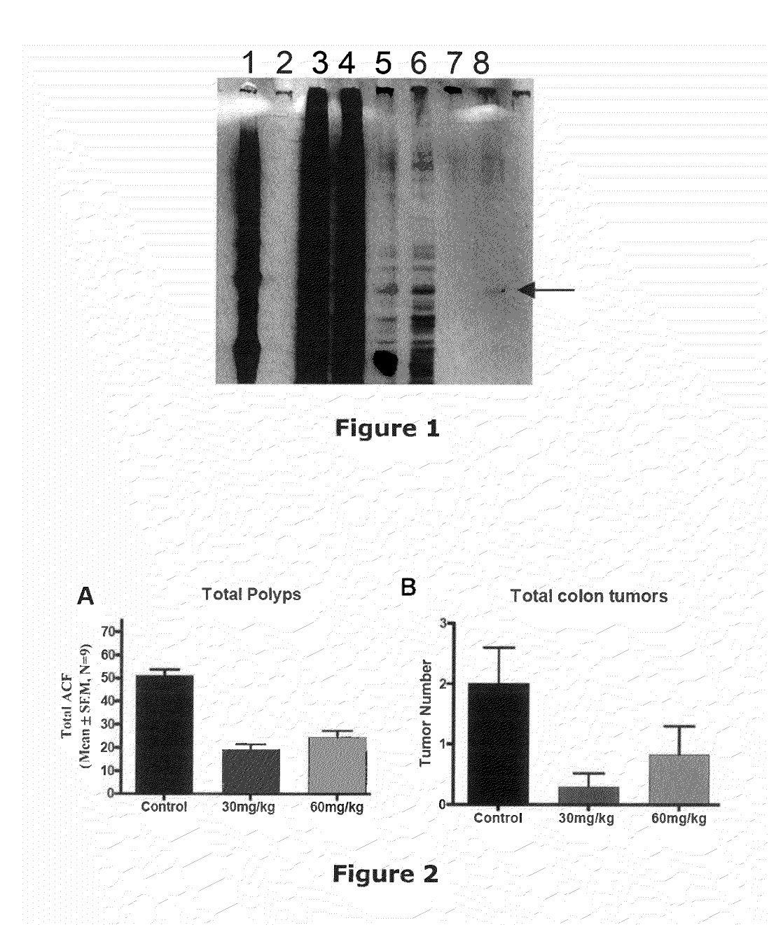

The presently disclosed inventive concept(s) is directed in one embodiment, to methods of treating and inhibiting several diseases, for example as described herein, by the administration of Flex-Hets, including for example but not by way of limitation, SHetA2, SHetA3, SHetA4, 5HetA19, SHetC2, and combinations thereof (see FIG. 1B of U.S. Pat. No. 7,612,107, the entire contents of which are expressly incorporated herein by reference).

Examples of the presently disclosed inventive concept(s) are provided hereinbelow. However, the presently disclosed inventive concept(s) is to be understood to not be limited in their application to the specific experimentation, results, and laboratory procedures shown herein. Rather, the examples are simply provided as several of various embodiments and are meant to be exemplary, not exhaustive.

Flex-Hets which may be used in the presently disclosed inventive concept(s) to bind to and/or modulate the activity of one, two, or all of HSPA5, HSPA8, and HSPA9 include, but are not limited to, those flexible heteroarotinoids described below and as shown in U.S. Pat. Nos. 6,586,460 and 7,612,107, which are hereby expressly incorporated by reference herein in their entireties.

Non-limiting examples of flexible heteroarotinoids that may be utilized in accordance with the methods of the presently disclosed and claimed inventive concept(s) include compounds of stereoisomeric Formulas (I) and (II):

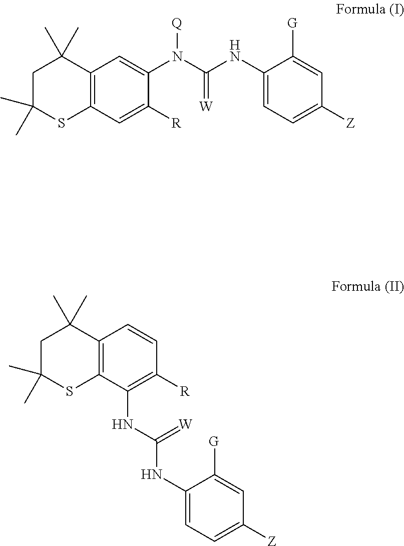

wherein R denotes H, CH₃, or OCH₃; Q denotes H or i-C₃H₇; W denotes O or S; G denotes H or CH₃; and Z denotes NO₂, CO₂Et, CO₂-n- C₄H₉, or SO₂NH₂.

Additional non-limiting examples of flexible heteroarotinoids that may be utilized in accordance with the methods of the presently disclosed and claimed inventive concept(s) include compounds of stereoisomeric Formulas (Ill) and (IV):

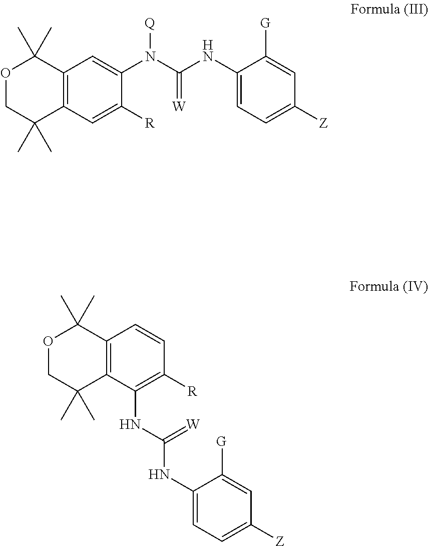

wherein R denotes H, CH₃, or OCH₃; Q denotes H or i-C₃H₇; W denotes O or S; G denotes H or CH₃; and Z denotes NO₂, CO₂Et, CO₂-n -C₄H₉, or SO₂NH₂.

Additional non-limiting examples of flexible heteroarotinoids that may be utilized in accordance with the methods of the presently disclosed and claimed inventive concept(s) include compounds of Formula (V):

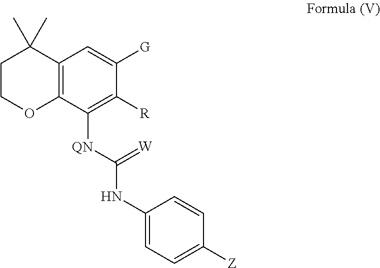

wherein R denotes H, CH₃, or OCH₃; Q denotes H or i-C₃H₇; W denotes O or S; G denotes H, CH₃, or C(O)—CH₃; and Z denotes NO₂, CO₂Et, or CO₂-n- C₄H₉.

Particular non-limiting examples of flexible heteroarotinoids that may be utilized in accordance with the methods of the presently disclosed and claimed inventive concept(s) include SHetA2, SHetA3, SHetA4, SHetA19, and SHetC2, and combinations thereof.

The Flex-Het SHetA2 has completed preclinical testing as a cancer chemoprevention agent at the National Institutes of Health (NIH), National Cancer Institute (NCI) Rapid Access to Preventive Intervention Development (RAPID) program. It has demonstrated cancer prevention activity without any toxicity, carcinogenicity or teratogenicity in multiple animal models. Magnetic beads with SHetA2 attached thereto were prepared. Conditions in which cancer cell extracts bound differentially to the SHetA2 beads in comparison to empty control beads were optimized. A Western blot was used to visualize and cut out the differential proteins, which were identified by mass spec analysis to be HSPA5, HSPA8, and HSPA9. All of the signaling pathways demonstrated to be regulated by Flex-Hets in cancer and polycystic kidney disease cells are also regulated by the HSPA5/8/9 proteins as shown in Table 1. Binding to these proteins was confirmed in a second repeat experiment.

The presently disclosed inventive concept(s), in particular, is directed to methods of treating multiple diseases described herein by administering flex-hets disclosed herein, which separately or simultaneously target or affect activity or expression of (1) HSPA5 (located in the ER lumen where it controls protein folding and ER stress/UPR/macroautophagy), (2) HSPA8 (located in the cytosol, where it controls protein folding, intracellular trafficking, cyclin D1 stability and function, and chaperone-mediated autophagy), and/or (3) HSPA9, (located primarily in the mitochondria where it controls import of proteins into the mitochondria and mitochondrial membrane integrity, but also located in the cytosol where it controls p53 function).

In one embodiment, the overall effect of the flex-hets on one, two, or all three of HSPA5, HSPA8, and HSPA9 is to stimulate survival responses in affected cells, for example by upregulating adaptive UPR and reducing ERS in the cells by decreasing protein synthesis, increasing synthesis of molecular chaperones to handle the overload, and reversing the flow of misfolded proteins back to the cytoplasm for proteasomal degradation. The response also involves, in certain diseases and conditions, induction of autophagy to clear damaged proteins and organelles. However, in cells that have become addicted to the survival proteins which slow down the cell proliferation and metabolism to allow repair, the pathways are shunted into programmed cell death. The ability to differentially affect diseased cells over normal cells has been demonstrated using Flex-Het drugs (10). Without being bound by theory, in one embodiment, the survival effect of SHetA2 appears to be related to the effect of disrupting HSPA8 with the cyclin D1 complex. SHetA2 disruption of the HSPA8 chaperone function on cyclin D1 induces cell cycle arrest in the G1 phase in both cancer and normal cells. The G1 arrest serves to protect normal cells, but is insufficient to protect diseased cells.

SHetA2 inhibition of HSPA8 stabilization of the cyclin D1 complex will induce a G1 cell cycle arrest that protects cells from SHetA2-induced stress pushing the cell into the apoptotic cell death pathway. In support of this, cells that do not have HSPA8 are more sensitive to SHetA2, cells that have developed resistant to SHetA2 have reduced cyclin D1 expression, and primary cultures of human mammary epithelial cells do not become sensitive to SHetA2 until after they have gone through a stasis (which upregulates p16 to inhibit cyclin D1) and into a post-stasis phase or immortal phase where p16 expression is lost. In each of the situations, cells are induced into a G1 arrest by SHetA2 interference with HSPA8/cyclin D1. In healthy cells, this G1 arrest protects the cell from cell death. The cancer cells that have reduced cyclin D1 are more resistant to SHetA2 induction of apoptosis, because they have already developed an inherent protective reduction of cyclin Dl. The human mammary epithelial cells that have lost p16, have essentially done the opposite of reducing cyclin D1, because by losing p16, they have essentially removed a cyclin D1 barrier, and are thus more sensitive to SHetA2 because SHetA2 has less ability to protect the cells through interference with HSPA8/cyclin D1.The specific molecular pathways mediating the Flex-Het effects are all down-stream signals known to mediate HSP/\5, HSPA8 and HSPA9 regulation of cellular fate (Table 1).

Discovery of HSPA5, HSPA8 and HSPA9 as SHetA2 binding proteins.

As mentioned above, to identify proteins which bind to SHetA2, whole cell protein extracts isolated from the human A2780 ovarian cancer cell line were incubated with NanoLink magnetic beads conjugated to SHetA2 (prepared by SoluLink, Inc., San Diego, Calif.). Empty NanoLink magnetic beads were used as a control, as noted previously. Solutions obtained through washing the beads and eluting with excess SHetA2 were evaluated on SDS gels. A 75 kD band was consistently observed in eluents from the SHetA2 beads and not in the control beads in 3 repetitions. Conditions of washing and eluting the proteins from the column were adjusted in the repetitions until sufficient amounts of the band could be obtained for mass spec analysis to identify the proteins (FIG. 1 and Table 3). An independent repeat experiment using the entire bead eluent instead of just the 75 kD band, identified the same three HSPA proteins as being bound by SHetA2.

TABLE 3
Mass Spec Identification of SHetA2 binding proteins in 75 kD band.

| Hit | Accession | Score | Mass | Av intensity | Name |
|---|---|---|---|---|---|
| 2 | gi\|5729877 | 1030 | 71082.81 | 628.0486 | HSPA8 |
| 3 | gi\|292059 | 423 | 74019.46 | 409.2786 | HSPA9 |
| 1 | gi\|6470150 | 1202 | 71002.07 | 312.8802 | HSPA5 |

Effects of SHetA2 on colorectal cancer and mammary cancer.

The in vivo chemoprevention activity of SHetA2 in a genetic colorectal cancer model (C57BU6J-APc^{min}) and a carcinogen-induced breast (mammary) cancer model were evaluated. In the colorectal cancer model, a SHetA2 diet significantly reduced tumor incidence and size in both male and female mice at doses of 30 and 60 mg/kg/day in comparison to control a control diet (FIG. 2) thereby demonstrating that SHetA2 is effective as a colorectal cancer treatment. There were no significant differences in weights of different treatment groups. Tumors of all sizes (<0.1 mm, between 0.1 and 0.2 mm, and >0.2 mm) and locations (duodenum, jejunum, and ileum) were significantly reduced by both drug concentrations in both sexes.

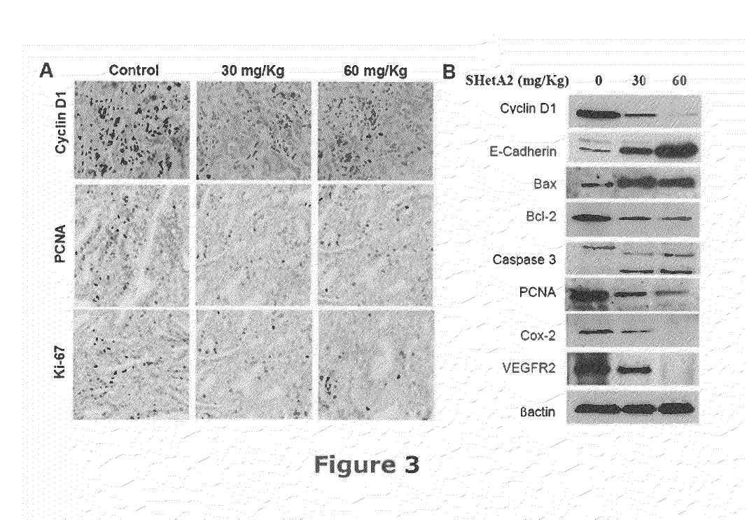

Based on mechanistic studies conducted in other cancer types, it was anticipated that the levels of cyclin D1, BcI-2, PCNA and Ki-67 would be decreased, while the levels of E-Cadherin and Bax would be increased, and Caspase 3 would be cleaved in SHetA2 treated tumors in comparison to controls. These molecular events, along with reduction of Cox-2 and VEGFR2, and other molecular targets in colorectal cancer are shown in FIG. 3.

An in vivo efficacy study found moderate in vivo anticancer activity in animals treated with SHetA2 in a chemically induced rat mammary cancer model (Table 4), demonstrating that SHetA2 is useful in mitigating mammary cancer.

TABLE 4
Effect of SHetA2 in Prevention of Methylnitrosourea (MNU)-induced Mammary
Cancers in Female Sprague-Dawley Rats

| | | | | Mammary Adenocarcinomas^{c} | | Avg.Weight of |
|---|---|---|---|---|---|---|
| Group | # Rats | Carcinogen^{a} | Treatment^{b} | % Incidence | Number/Rat | Cancers |
| 1 | 15 | MNU | SHetA2 150 | 87 | 2.8 (32% ↓)^{d} | 6.0 (35% ↓) |
| | | | mg/kg BW/day | | | |
| 2 | 15 | MNU | SHetA2 50 | 80 | 2.1 (49% ↓) | 5.1 (45% ↓) |
| | | | mg/kg BW/day | | | |
| 3 | 15 | MNU | No Treatment | 93 | 4.1 | 9.3 |

^{a}Female Sprague-Dawley rats received MNU at 50 days of age.
^{b}SHetA2 was administered beginning when the rats were 55 days of age.
^{c}Data on mammary cancers were obtained at necropsy of the rats (126 days after MNU).
^{d}Numbers in parenthesis are percent differences from control group.

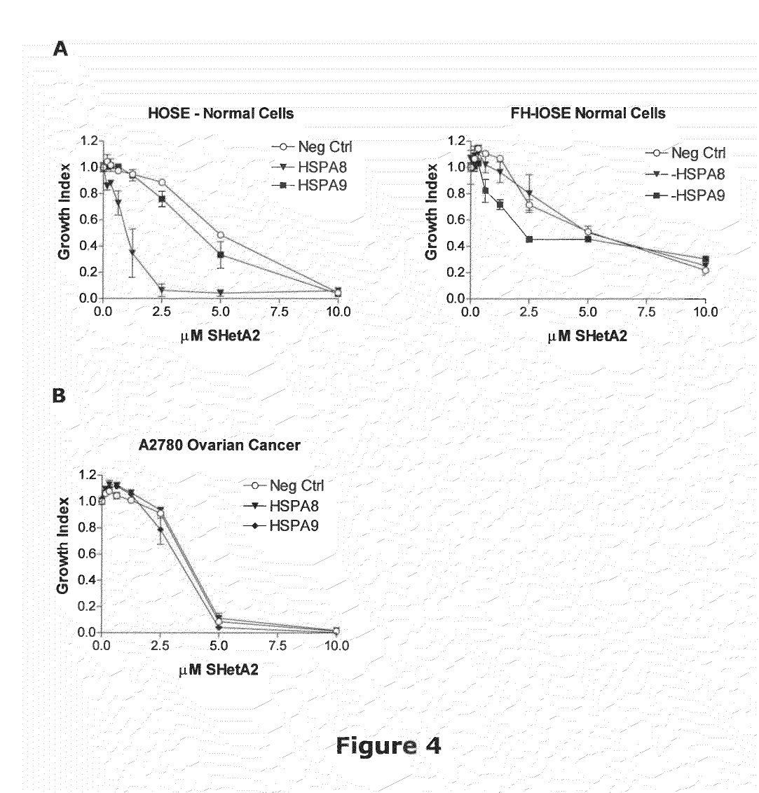

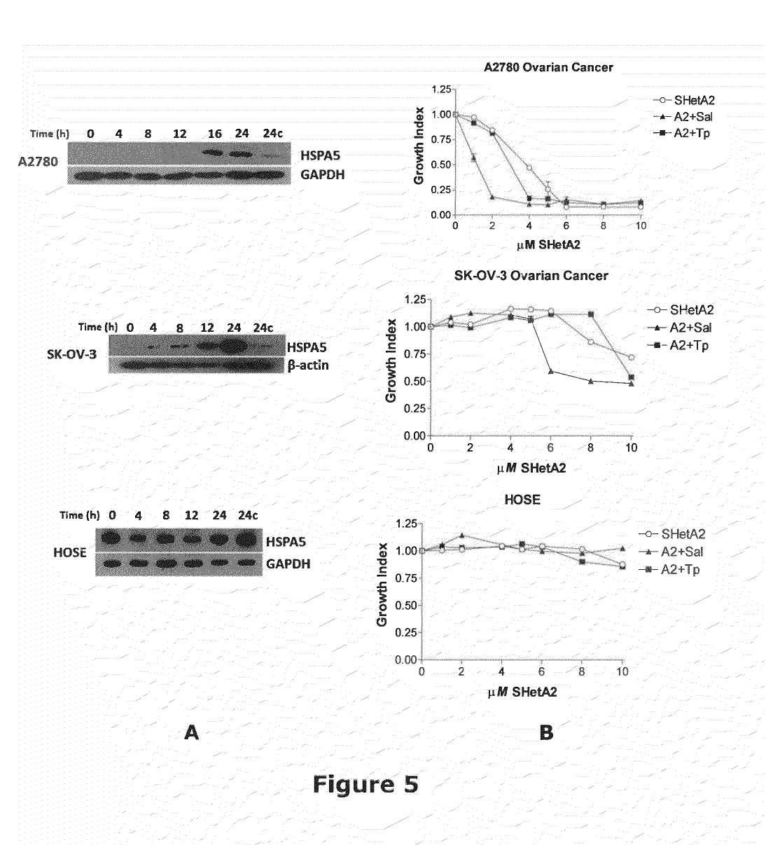

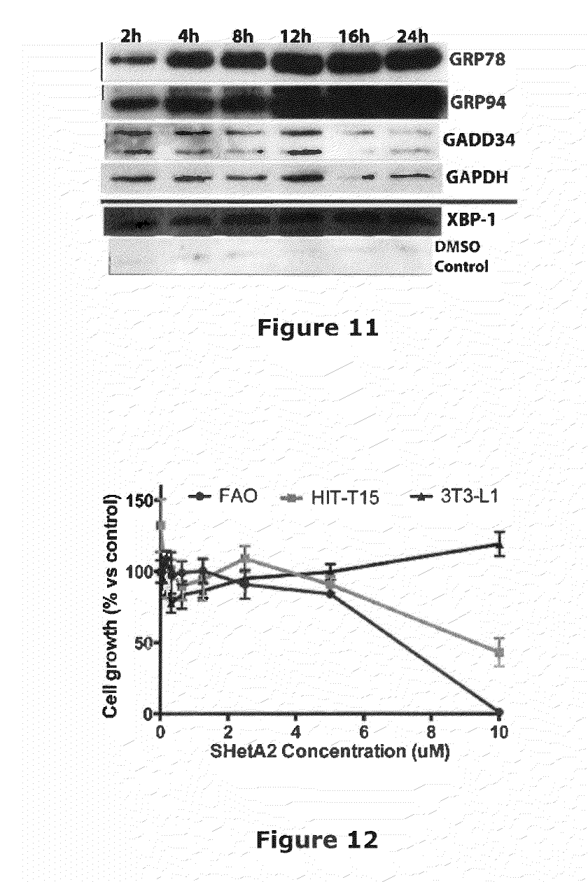

Based on the presently disclosed inventive concept(s), it was concluded that the HSPA proteins initiate the known SHetA2 molecular mechanisms, however, it was desired that the functional link be further validated experimentally. The requirement of each HSPA protein in the mechanism of Flex-Het protection of normal cells, but not diseased cancer cells, was tested by determining if loss of expression using Silencer Select Pre-designed siRNAs (Applied Biosystems/Ambion) prevented the growth inhibition. Reduction of HSPA8 with an siRNA eliminated the protective effect of SHetA2 on normal human ovarian surface epithelial cells (FIG. 4A), while growth inhibition by SHetA2 in cancer cells was not affected by the HSPA5, HSPA8, or HSPA9 siRNAs ̂ Reduction of HSPA9 with siRNA reduced the protective effect of SHetA2 in human ovarian surface epithelial cells from individuals with a genetic predisposition for cancer making them more dependent on HSPA9 for preventing the programmed cell death pathway induced by p53 (FIG. 4B). These results demonstrate that upon the reduction of the expression of HSPA8 and HSPA9 proteins individually, the ability of SHetA2 to protect cells is reduced, thus demonstrating that SHetA2 requires these proteins to prevent the toxic effects of SHetA2 in normal cells. High dose (10 μM) SHetA2 induces expression of HSPA5 in diseased cancer cells, but not in healthy cells (FIG. 5A). When the “adaptive response” is inhibited with salubrinol, the selective cytotoxicity of high dose SHetA2 on diseased cells over normal cells is reduced, indicating that the induction of HSPA5 leads to an “adaptive response” that counteracts cell death (FIG. 5B) in the absence (SHetA2) or presence of salubrinol (A2+Sal) to inhibit the “adaptive response” or Thapsigargin (A2+Tp) to induce ER stress in triplicate. The growth index was determined with the MIS cytotoxicity assay by dividing the optical density (OD) output of the assay for the treated cultures by the average (OD) of control cultures treated with solvent (dimethyl sulfoxide) only. When a low dose (e.g., 1 μM) of SHetA2 is administered to subjects suffering from diabetes, the induction of HSPA5 can lead to an “adaptive response” which can help to clear pancreatic islet amyloid, promote normal production of insulin granules and prevent apoptosis. FIG. 11 (discussed further below) shows that SHetA2 is not cytotoxic to FAO liver cells, differentiated 3T3-L1 adipocytes, and HIT-T15 β-cells at low levels (up to 5 μM). Toxicity to the liver cells and beta cells increases at higher levels (10 μM).

SHetA2-induced Autophagy.

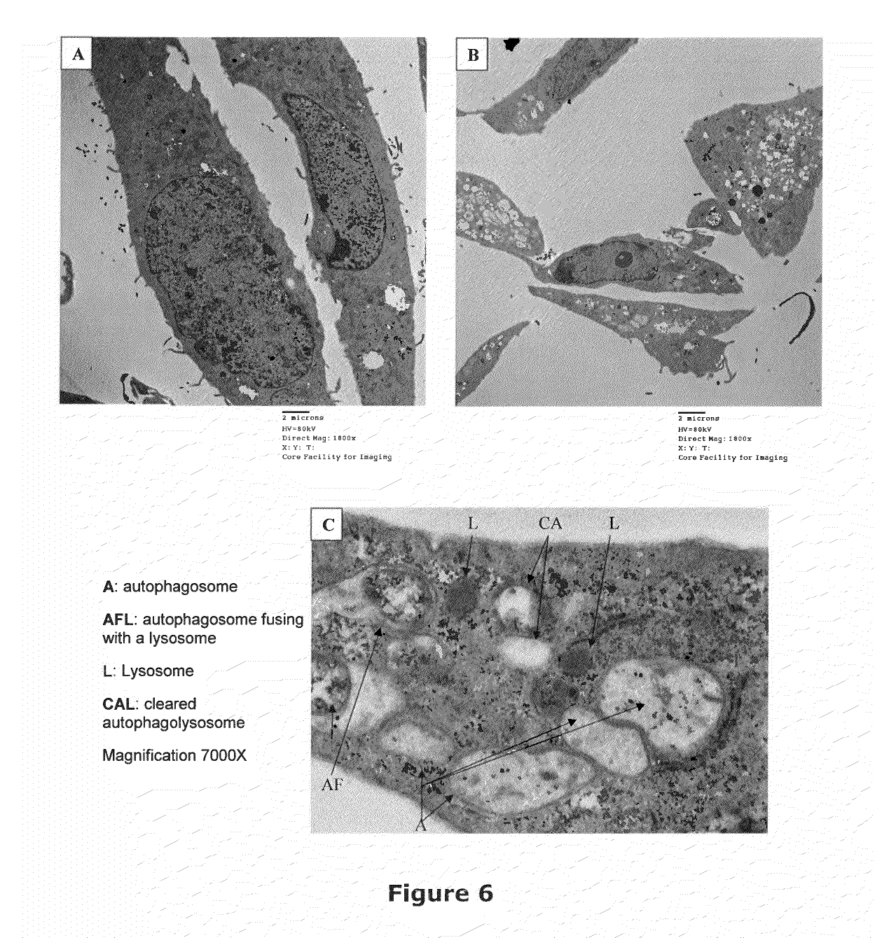

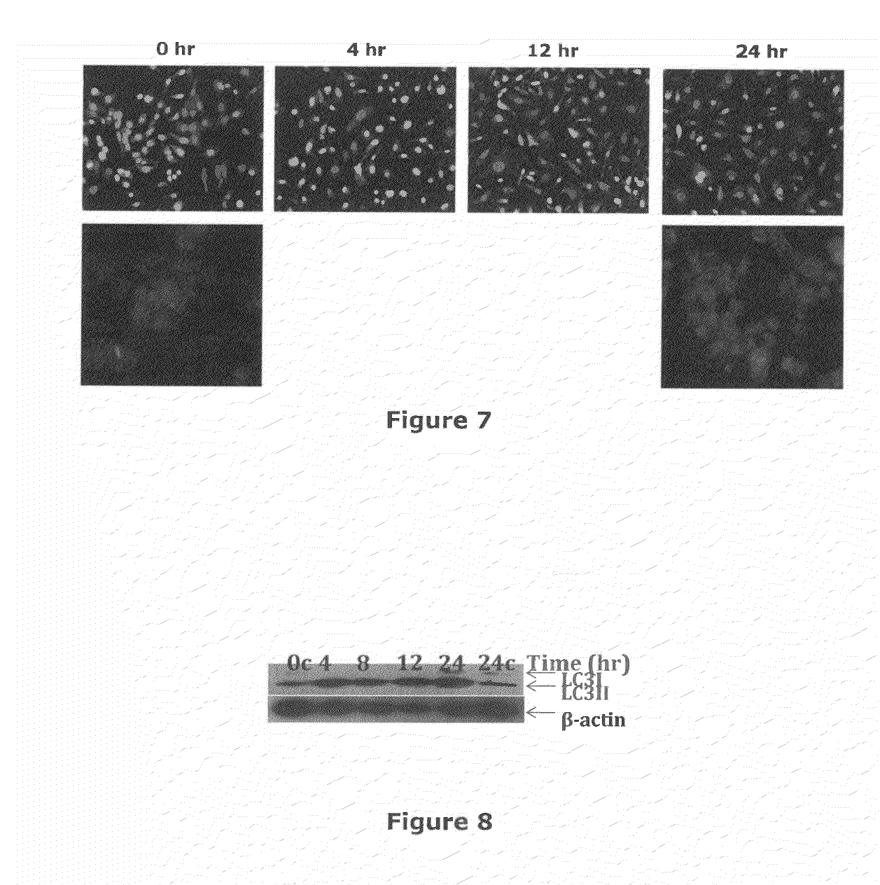

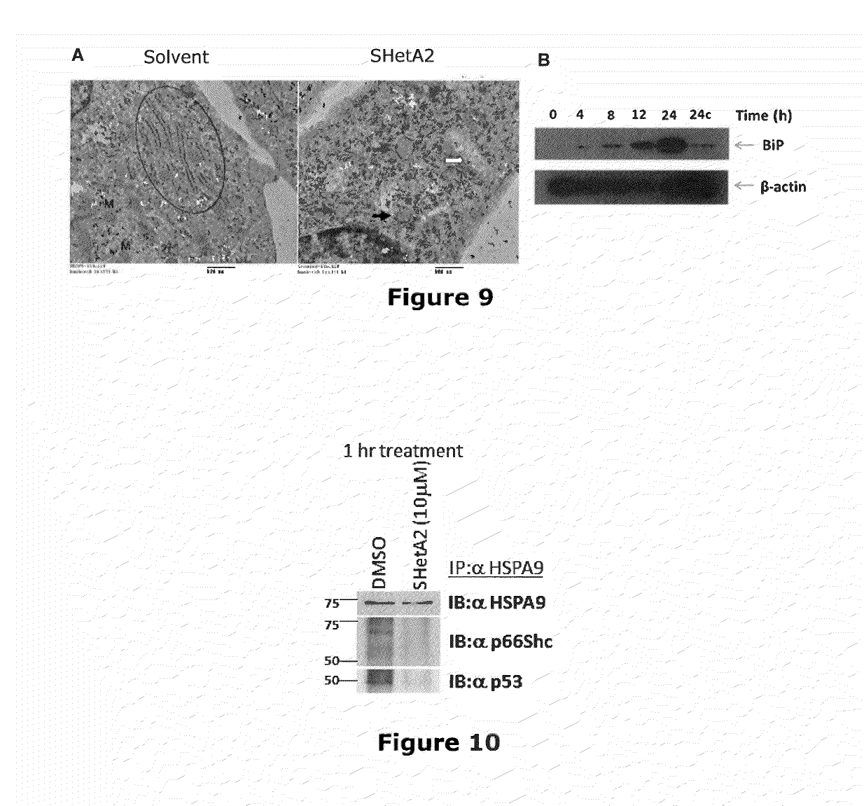

At certain concentrations, SHetA2 induces both ER stress and autophagy. FIG. 6 shows cleared vesicles and smaller sizes of the cells treated with SHetA2 in comparison to the solvent only-treated controls, demonstrating that the process of autophagy is not blocked, but instead pushes through to complete digestion/clearing of the lysosomal contents. FIG. 6 thus shows that SHetA2 can induce autophagy in diseased cells through the complete processing resulting in degradation of the defective proteins in lysosomes. FIGS. 7 and 8 provide further evidence for SHetA2 induction of autophagy. FIG. 7 demonstrates increases of acridine orange staining of vesicles in diseased cancer cells treated with SHetA2 increase over time, while there is little induction in normal health cells. Acridine orange is a lysosomo-tropic agent, that can penetrate biological membranes when uncharged. The protonated form accumulates in acidic compartments, where it forms aggregates that fluoresce bright red. Thus it is used to measure autophagolysosomes and lysosomes. A biochemical assay that confirms the process of autophagy is occurring in cells is Western blot measurement of light chain 3 (LC3). When autophagy is induced, LC3I is conjugated to phosphatidylethanolamine resulting in a form of LC3, called LC3-II, which has a higher electrophoretic mobility in SDS page gels than the LC3I form. LC3-II is the only protein known to be specifically localized to autophagic vesicles throughout the process of autophagosome formation to lysosomal degradation (13). FIG. 8 demonstrates the induction of LC3-II by SHetA2 treatment of diseased ovarian cancer cells. FIG. 9 demonstrates that SHetA2 provided at 10 μM induces ER stress and HSPA5 (BiP) expression in ovarian cancer cells.

The ability of SHetA2 to interfere with HSPA9 function was demonstrated by showing that SHetA2 can prevent HSPA9 binding to its client proteins. FIG. 10 demonstrates that SHetA2 provided at 10 μM interferes with HSPA9 interaction with its substrate proteins, p66Shc and p53. HSPA9 regulates the ability of the p66 Shc protein to induce senescence (14) and apoptosis (15). HSPA9 regulates the subcellular distribution of p53 thereby controlling p53 induction of apoptosis (16). SHetA2 inhibition of HSPA9 interaction with p66shc causes mitochondrial damage that increases the level of autophagy. Autophagy is a process whereby membranes form de novo inside of cells surrounding entire sections of cytoplasm including organelles. In SHetA2 treated cells, the damaged mitochondria disappear and their remnants can be seen inside of autophagosomes. The autophagosomes are capable of clearing the accumulated proteins in the diseases listed herein.

The ability of low levels of SHetA2 to induce adaptive UPR and do not convert into apoptotic cell death is shown in FIGS. 11 and 12. FIG. 11 demonstrates that a concentration of 1 μM SHetA2 can induce expression of UPR biomarker proteins ((HSPA5 (GRP78), GRP94, GADD34, XBP-1)) in liver cells. FIG. 12 demonstrates that SHetA2 concentrations at or below 5 μM do not cause cytotoxicity in liver cells or in undifferentiated or differentiated fat cell.

Utility

The presently disclosed inventive concept(s) is directed to treatment of diseases or conditions involving defective protein folding, in particular those associated with ERS, by administering to a subject having such disease or condition, one or more flexible heteroarotinoids as described herein. The diseases or conditions which can be treated as described herein include, but are not limited to: cancers (including, but not limited to, colorectal, mammary, and ovarian cancer), polycystic kidney disease (PKD), bipolar disorder, obesity, insulin resistance, diabetes mellitus (T1DM and T2DM), hyperinsulenima (pre-diabetes), atherosclerosis, inflammation, ischemia, heart diseases, liver diseases, kidney diseases, viral infection, hereditary tyrosinemia type I, and neurodegenerative diseases,

Wolcott-Rallison Syndrome, Wolfram Syndrome, familial hypercholesterolemia, Z alphal-antitrypsin deficiency, Inclusion body myopathy (IBMPFD), Parkinsons disease, Familial Alzheimer's disease, Familial amyotrophic lateral sclerosis, Marinesco-Sjogren syndrome, Pelizaeus-Merzbacher disease, Transmissible spongiform encephalopathy, spinocerebellar ataxia 3/Machado-Joseph disease, Huntington's disease, Sproadic inclusion body myositis, Cerebral ischemia, fluoride tooth, schizophrenia, and lysosomal storage diseases (see below).

In one embodiment, the method of presently disclosed inventive concept(s) is directed to treating in a subject a disease or condition characterized by or associated with at least one of (1) endoplasmic reticulum (ER) stress, (2) a cellular accumulation of unfolded or misfolded proteins, and (3) an abnormal unfolded protein response, comprising: administering to the subject a composition comprising a flexible heteroarotinoid leading to a reduction in ERS in a cell, reduction in the accumulation of unfolded or misfolded proteins in a cells, or an apoptotic response in cells leading to death of the cells in the subject.

Examples of types of cancer which can be treated by the Flex-Hets disclosed herein include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, mammary cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

Any inflammatory condition known in the art or otherwise contemplated herein may be treated in accordance with the presently disclosed inventive concept(s). Non-limiting examples of diseases or conditions having inflammation associated therewith include infection-related or non-infectious inflammatory conditions in the lung (i.e., sepsis, lung infections, Respiratory Distress Syndrome, bronchopulmonary dysplasia, etc.); infection-related or non-infectious inflammatory conditions in other organs (i.e., colitis, Inflammatory Bowel Disease, diabetic nephropathy, hemorrhagic shock); inflammation-induced cancer (i.e., cancer progression in patients with colitis or Inflammatory Bowel Disease); and the like.

Examples of lysosomal storage diseases which can be treated with the Flex-Hets described herein include, but are not limited to, Activator Deficiency/GM2 gangliosidosis, alpha-mannosidosis, aspartylglucosaminuria, cholesteryl ester storage disease, cystinosis, Danon disease, Fabry disease, Farber disease, fucosidosis, galactosialidosis, Gaucher disease (Types I, II, II), GM1 gangliosidosis (infantile, late infantile/juvenile, adult/chronic), I-cell disease/Mucolipidosis, Infantile free sialic acid storage disease/ISSD, Juvenile hexosaminidase A deficiency, Krabbe disease (infantile onset, late onset), lysosomal acid lipase deficiency (early onset/late onset), metachromatic leukodystrophy, Pseudo-Hurler polydystrophy/mucolipidosis IIIA, mucopolysaccharidosis I (MPS I) Hurler syndrome, MPS I Scheie syndrome, MPS I Hurler-Scheie syndrome, MPS II Hunter syndrome, Sanfilippo syndrome Type A (MPS IIIA), Sanfilippo syndrome Type B (MPS IIIB), Sanfilippo syndrome Type C (MPS IIIC), Sanfilippo syndrome Type D (MPS IIID), Morquio Type A/MPS IVA, Morquio Type B/MPS IVB, MPS IX hyaluronidase deficiency, MPS VI Maroteaux-Lamy, MPS VII Sly syndrome, mucopolylipidosis I/sialidosis, mucolipidosis IIIC, mucolipidosis type IV(multiple sulfatase deficiency, Niemann-Pick disease (Types A, B, C), CLN6 disease (atypical late infantile, late onset variant, early juvenile), Batten-Spielmeyer-Vogt/Juvenile NCL/CLN3 disease, Finnish Variant late infantile CLN5, Jansky-Bielschosky disease/late infantile CLN2/TPP1 disease, Kufs/Adult-onset NCL/CLN4 disease, Northern epilepsy/variant late infantile CLN8, Santavuori-Haltia/infantile CLN1/PPT disease, beta-mannosidosis, Pompe disease/glycogen storage disease type II, pycnodysostosis, Sandhoff disease/GM2 gangliosidosis (adult onset, infantile onset, juvenile onset), Schindler disease, Sall disease/sialic acid storage disease, Tay-Sachs/GM2 gangliosidosis, and Wolman disease.

HSPA5 and Endoplasmic Reticulum Stress

HSPA5 is known to modulate how cells respond to ERS. ERS has been known as a key factor in the etiopathogenesis of various diseases for decades yet has become a major focus in the diabetes field in the past approximately two years. This has been highlighted by a paper by Ozcan, et al. (Science (2006) 313:1137-1140) which showed a link between chaperone modulation of ERS and glucose homeostasis in a T2DM mouse model. As explained elsewhere herein, cells respond to ERS by activating assorted pathways which include, in part, the Unfolded Protein Response (UPR). UPR activation tries to reduce ERS (“adaptive” UPR) and, if unsuccessful, will ultimately lead to activation of factors which stimulate cell death pathways. HSPA5 is viewed as the “master regulator” of stimulating UPR response pathways in the cell. It is for this reason that HSPA5 is a major focus for many labs and pharmaceutical development entities for the development of small molecule therapeutics which modulate HSPA5 activation towards a specific outcome.

T2DM generally progresses through several phases from pre-diabetes to full-blown insulin dependence. As elevated glucose levels occur in the body, it will place a higher demand on insulin secreting pancreatic beta-cells to produce insulin and restore glucose homeostasis (or alleviate postprandial spikes). This increased demand to fold insulin leads to ERS in the beta cells. ERS can also result in phosphorylation of the insulin receptor which attenuates insulin efficacy causing insulin resistance and, as a result, further increases the demand for insulin production. As this ERS in the beta cells continues it will eventually lead to a loss in beta cell mass which has been observed in autopsies of T2DM individuals. Since beta cells do not appear to be regenerated in the pancreas, this loss in beta cell mass leads to the fully insulin-dependent phenotype of later stage T2DM patients.

In the presently disclosed inventive concept(s), it has been shown that low doses of Flex-Hets, e.g., SHetA2, can stimulate the adaptive UPR pathway without inducing apoptosis. Activation of the adaptive UPR in a diabetic patient is beneficial because it (1) reduces ERS and thus increases insulin sensitivity in peripheral tissues by preventing IRS-1 phosphorylation, (2) improves insulin production by attenuating ERS, (3) decreases insulin resistance, and (4) will preserve pancreatic beta cells thus maintaining beta cell mass due to amelioration of ERS. The latter serves to prevent one of the most deleterious outcomes in T2DM which is loss of beta cell mass. In regard to T1DM, amelioration of beta cell loss during the early stages of the diseases helps preserve beta cell mass while treatments against beta cell-killing autoimmunity of T1DM are mounted. A low dosage is considered in one embodiment to be a dosage that results in an average blood concentration of less than or equal to about 5 μM.

Therefore, one embodiment of the presently disclosed inventive concept(s) is a method of preserving beta cell mass in a subject by treating the subject with a Flex-Het as disclosed herein. The preservation of beta cell mass in the subject can be shown by an increase or stabilization in the amount of insulin production and/or C-peptide production in the subject (as measured by blood or urine tests). In one suitable assay, beta cell mass is indirectly calculated by determining the ratio of C-peptide-to-glucose following oral glucose ingestion, preferably as measured 15 minutes after glucose ingestion (Meier at al., Diabetes 2009; 58:1595-1603). Alternatively, beta cell mass preservation can be indirectly calculated by using the Homeostatsis model assessment (HOMA) index (Matthews et al., Diabetologia 1985; 28: 412-419). Additionally, in one embodiment, the treatment with a Flex-Het described herein in a subject having diabetes results in a blood hemoglobin A1[ value which is less than about 7% in the subject. Also, as noted above, amylin forms aggregrates pancreatic islet beta cells in individuals with T2DM. In one embodiment, the Flex-Hets described herein (e.g., SHetA2) can be used to treat diabetes by inducing autophagy and removal of amylin aggregates in beta cells.

A therapeutically effective amount of the Flex-Het as described herein used in the treatment described herein can be readily determined by the attending diagnostician, as one skilled in the art, by the use of conventional techniques and by observing results obtained under analogous circumstances. In determining the therapeutically effective dose, a number of factors are considered by the attending diagnostician, including, but not limited to: the species of mammal; its size, age, and general health; the specific disease or condition involved; the degree of or involvement or the severity of the disease or condition; the response of the individual subject; the particular compound administered; the mode of administration; the bioavailability characteristic of the preparation administered; the dose regimen selected; the use of concomitant medication; and other relevant circumstances. A therapeutically effective amount of a compound of the presently disclosed inventive concept(s) also refers to an amount of a Flex-Het which is effective in controlling or reducing the disease or condition. The amount of the Flex-Het in the pharmaceutical composition of the presently disclosed inventive concept(s) will depend upon the nature and severity of the condition being treated, and on the nature of prior treatments which the patient has undergone. Ultimately, the attending physician will decide the amount of Flex-Het with which to treat each individual patient. Initially, the attending physician will preferably administer low doses of the Flex-Het and observe the patient's response. Larger doses may be administered until the optimal therapeutic effect is obtained for the patient, and at that point the dosage is not increased further.

Administration of the Flex-Het used in the pharmaceutical composition or to practice the method of the presently disclosed inventive concept(s) can be carried out in a variety of conventional ways, such as, but not limited to, orally, by inhalation, rectally, or by cutaneous, subcutaneous, intraperitoneal, vaginal, or intravenous injection. Oral formulations may be formulated such that the Flex-Het passes through a portion of the digestive system before being released, for example it may not be released until reaching the small intestine.

When a therapeutically effective amount of the Flex-Het is administered orally, the compound may be in the form of a tablet, capsule, powder, solution or elixir. The pharmaceutical composition may additionally contain a solid carrier such as a gelatin or an adjuvant. The tablet, capsule, and powder preferably contains from about .05 to 95% of the Flex-Het by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, 35 propylene glycol, or polyethylene glycol. When administered in liquid form, the pharmaceutical composition preferably contains from about 0.005 to 95% by weight of the Flex-Het. For example, a dose of 10-1000 mg once to twice a day could be administered orally.

For oral administration, the Flex-Het can be formulated into solid or liquid preparations such as capsules, pills, tablets, lozenges, melts, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

When a therapeutically effective amount of the Flex-Het is administered by intravenous, cutaneous or subcutaneous injection, the Flex-Het is preferably in the form of a pyrogen-free, parenterally acceptable aqueous solution or suspension. The preparation of such parenterally acceptable solutions, having due regard to pH, isotonicity, stability, and the like, is within the skill in the art. A preferred pharmaceutical composition for intravenous, cutaneous, or subcutaneous injection may contain, in addition to the Flex-Het, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the presently disclosed inventive concept(s) may also contain stabilizers, preservatives, buffers, antioxidants, or other additive known to those of skill in the art.

As noted above, the compositions can also include an appropriate carrier. For topical use, any of the conventional excipients may be added to formulate the Flex-Het into a lotion, ointment, powder, cream, spray, or aerosol. For surgical implantation, the Flex-Het may be combined with any of the well-known biodegradable and bioerodible carriers, such as polylactic acid and collagen formulations. Such materials may be in the form of solid implants, sutures, sponges, wound dressings, and the like. In any event, for local use of the materials, the Flex-Het is usually present in the carrier or excipient in a weight ratio of from about 1:10 to 1:20,000, but is not limited to ratios within this range. Preparations of compositions for local use are detailed in Remington: The Science and Practice of Pharmacy, 21^{st} ed.

The presently disclosed inventive concept(s) further includes a method of treatment by topically applying an amount of the Flex-Het. The topical medication may take any number of standard forms such as pastes, gels, creams, and ointments. Topical application may be accomplished by simply preparing a solution of the Flex-Het to be administered, preferably using a solvent known to promote transdermal absorption such as ethanol or dimethyl sulfoxide (DMSO) with or without other excipients. Preferably topical administration will be accomplished using a patch either of the reservoir and porous membrane type or of a solid matrix variety.

Therapeutically effective amounts of the compositions of the Flex-Hets as described herein will generally be from about 0.01 μg/kg to about 1000 mg/kg (weight of active ingredient/body weight of patient). Preferably, the dosage will deliver at least 0.1 μg/kg to 100 g/kg, and more preferably at least 1 mg/kg to 10 mg/kg of the Flex-Het as described herein. In preferred embodiments the dosage will be formulated to maintain an average blood concentration of at least one of about .1 μM, about .5 μM, about 1 μM, about 2 μM, about 3 μM, about 4 μM, about 5 μM, about 6 μM, about 7 μM, about 8 μM, about 9 μM, and about 10 μM of the Flex-Het as described herein.

Practice of the methods of the presently disclosed inventive concept(s) comprises administering to a subject a therapeutically effective amount of the Flex-Nets as described herein in any suitable systemic or local formulation, in an amount effective to deliver the dosages listed herein. The dosage can be administered on a one-time basis, or (for example) from one to five times per day or once or twice per week, or continuously via a venous drip, depending on the desired therapeutic effect. The Flex-Hets as described herein can be used in preparations that contain additional pharmaceuticals for combination therapies, and can be used in combination with other pharmaceuticals administered separately.

As noted, particular amounts and modes of administration are able to be determined by one skilled in the art. One skilled in the art of preparing formulations can readily select the proper form and mode of administration depending upon the particular characteristics of the Flex-Nets as described herein selected, the disease state to be treated, the stage of the disease, and other relevant circumstances using formulation technology known in the art, described, for example, in Remington: The Science and Practice of Pharmacy, 21^{st} ed.

The Flex-Het compounds described herein may be used as a pharmaceutical composition when combined with a pharmaceutically acceptable carrier. Such a composition may contain, in addition to the Flex-Het and carrier, diluents, fillers, salts, buffers, stabilizers, solubilizers, and other materials well known in the art. Suitable carriers, vehicles and other components of the formulation are described, for example, in Remington: The Science and Practice of Pharmacy, 21^{st} ed. The term “pharmaceutically acceptable” means a non-toxic material that does not interfere with the effectiveness of the biological activity of the active ingredient(s). The characteristics of the carrier will depend on the route of administration.

The pharmaceutical composition of the presently disclosed inventive concept(s) may be in the form of a liposome in which Flex-Het is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

Pharmaceutical compositions can be manufactured utilizing techniques known in the art. Typically the therapeutically effective amount of the Flex-Het will be admixed with a pharmaceutically acceptable carrier. The compositions of the Flex-Hets of the presently disclosed inventive concept(s) may be administered by a variety of routes, for example, topically, orally, or parenterally (i.e., subcutaneously, intravenously, intramuscularly, intraperitoneally, or intratracheally). For oral administration, the Flex-Het can be formulated into solid or liquid preparations such as capsules, pills, tablets, lozenges, melts, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

In another embodiment, the Flex-Hets of the presently disclosed inventive concept(s) can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active ingredient in an aqueous or non-aqueous pharmaceutically acceptable solvent which may also contain suspending agents, sweetening agents, flavoring agents, and preservative agents as are known in the art.

For parenteral administration, the Flex-Hets may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives, and buffers as are known in the art.

The Flex-Hets of the presently disclosed inventive concept(s) can also be administered topically. This can be accomplished by simply preparing a solution of the compound to be administered, preferably using a solvent known to promote transdermal absorption such as ethanol or dimethyl sulfoxide (DM50) with or without other excipients. Preferably topical administration will be accomplished using a patch either of the reservoir and porous membrane type or of a solid matrix variety.

When the Flex-Het is administered intravenously, the duration of intravenous therapy using the pharmaceutical composition of the presently disclosed inventive concept(s) will vary, depending on the severity of the disease being treated and the condition and potential idiosyncratic response of each individual patient. It is contemplated that the duration of each application of the Flex-Het may be in the range of 1 to 2 hours and given once every 12 or 24 hours by continuous intravenous administration. Other antibiotics, intravenous fluids, cardiovascular and respiratory support could also be provided if requested by the attending physician in a manner known to one of ordinary skill in the art.

Additional pharmaceutical methods may be employed to control the duration of action or duration of release into the bloodstream of the Flex-Het. Increased half-life and controlled release preparations may be achieved through the use of polymers to conjugate, complex with, or absorb the Flex-Het described herein. The controlled delivery and/or increased half-life may be achieved by selecting appropriate macromolecules (for example, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide, and the appropriate concentration of macromolecules as well as the methods of incorporation, in order to control release.

Another possible method useful in controlling the duration of action by controlled release preparations and half-life is incorporation of the Flex-Het into particles of a polymeric material such as polyesters, polyamides, polyamino acids, hydrogels, poly(lactic acid), ethylene vinylacetate copolymers, copolymer micelles of, for example, PEG and poly(I-aspartamide).

It is also possible to entrap the Flex-Het in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art (Remington: The Science and Practice of Pharmacy, 21^{st} ed.).

As noted above, the compositions can also include an appropriate carrier. For topical use, any of the conventional excipients may be added to formulate the active ingredients into a lotion, ointment, powder, cream, spray, or aerosol. For surgical implantation, the active ingredients may be combined with any of the well-known biodegradable and bioerodible carriers, such as polylactic acid and collagen formulations. Such materials may be in the form of solid implants, sutures, sponges, wound dressings, and the like. In any event, for local use of the materials, the active ingredients usually be present in the carrier or excipient in a weight ratio of from about 1:1000 to 1:2[,000, but are not limited to ratios within this range. Preparations of compositions for local use are detailed in Remington: The Science and Practice of Pharmacy, 21^{st} ed.

The half-lives of the Flex-Hets described herein can be extended by their being conjugated to other molecules such as polymers using methods known to persons of ordinary skill in the art to form drug-polymer conjugates. For example, the Flex-Hets can be bound to molecules of inert polymers known in the art, such as a molecule of polyethylene glycol (PEG) in a method known as “PEGylation”. PEGylation can therefore extend the in vivo lifetime and thus therapeutic effectiveness of the Flex-Hets. PEGylation also reduces the potential antigenicity of the Flex-Hets. PEGylation can also enhance the solubility of Flex-Hets thereby improving their therapeutic effect. PEGs used may be linear or branched-chain.

By “PEGylated Flex-Het” is meant a Flex-Het of the presently disclosed inventive concept(s) having a polyethylene glycol (PEG) moiety covalently bound to a linking group of the Flex-Het. By “polyethylene glycol” or “PEG” is meant a polyalkylene glycol compound or a derivative thereof, with or without coupling agents or derivatization with coupling or activating moieties (e.g., with thiol, triflate, tresylate, azirdine, oxirane, or preferably with a maleimide moiety). Compounds such as maleimido monomethoxy PEG are exemplary or activated PEG compounds of the invention. Other polyalkylene glycol compounds, such as polypropylene glycol, may be used in the present invention. Other appropriate polymer conjugates include, but are not limited to, non-polypeptide polymers, charged or neutral polymers of the following types: dextran, colominic acids or other carbohydrate based polymers, biotin derivatives and dendrimers, for example. The term PEG is also meant to include other polymers of the class polyalkylene oxides.

The chemically modified Flex-Hets contain at least one PEG moiety, preferably at least two PEG moieties, up to a maximum number of PEG moieties bound to the Flex-Het without abolishing activity. The PEG moiety attached to the Flex-Hets may range in molecular weight from about 200 to 20,000 MW. Preferably the PEG moiety will be from about 1,000 to 8,000 MW, more preferably from about 3,250 to 5,000 MW, most preferably about 5,000 MW. The actual number of PEG molecules covalently bound per chemically modified Flex-Het of the invention may vary widely depending upon the desired Flex-Het stability (i.e. serum half-life).

U.S. Pat. No. 4,789,734 describes methods for encapsulating biochemicals in liposomes and is hereby expressly incorporated by reference herein. Essentially, the Flex-Het is dissolved in an aqueous solution, the appropriate phospholipids and lipids added, along with surfactants if required, and the material dialyzed or sonicated, as necessary. A review of known methods is by G. Gregoriadis, Chapter 14. “Liposomes”, Drug Carriers in Biology and Medicine, pp. 287-341 (Academic Press, 1979). Microspheres formed of polymers or proteins are well known to those skilled in the art, and can be tailored for passage through the gastrointestinal tract directly into the blood stream. Alternatively, the agents can be incorporated and the microspheres, or composite of microspheres, implanted for slow release over a period of time, ranging from days to months. See, for example, U.S. Pat. Nos. 4,90,6,474; 4,925,673; and 3,625,214 which are incorporated by reference herein.

When the Flex-Het composition is to be used as an injectable material, it can be formulated into a conventional injectable carrier. Suitable carriers include biocompatible and pharmaceutically acceptable phosphate buffered saline solutions, which are preferably isotonic. For reconstitution of a lyophilized product in accordance with the presently disclosed inventive concept(s), one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition which contains the Flex-Het may also be in the form of an aqueous solution containing many of the same substances as described above for the reconstitution of a lyophilized product.

The Flex-Het can also be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, nnalonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

As mentioned above, the Flex-Hets of the presently disclosed inventive concept(s) may be incorporated into pharmaceutical preparations which may be used for therapeutic purposes. However, the term “pharmaceutical preparation” is intended in a broader sense herein to include preparations containing a Flex-Het composition in accordance with this invention, used not only for therapeutic purposes but also for reagent or diagnostic purposes as known in the art, or for tissue culture. The pharmaceutical preparation intended for therapeutic use should contain a “pharmaceutically acceptable” or “therapeutically effective amount” of a Flex-Het, i.e., that amount necessary for preventative or curative health measures. If the pharmaceutical preparation is to be employed as a reagent or diagnostic, then it should contain reagent or diagnostic amounts of a Flex-Het.

In one embodiment, the presently disclosed inventive concept is a method of treating a diabetic or pre-diabetic condition in a subject in need of such treatment, comprising administering to the subject a therapeutically-effective amount of a flexible heteroarotinoid as defined by at least one of Formulas (I), (II), (III), (IV), and (V). In particular non-limiting embodiments, the flexible heteroarotinoid may be selected from SHetA2, SHetA3, SHetA4, SHetA19, and SHetC2, and combinations thereof. The method may be a treatment which results, in the subject, in at least one of (1) an increase in insulin sensitivity, (2) a decrease in insulin resistance, (3) a decrease in blood glucose levels, (4) a stabilization or increase in C-peptide production, e.g., as indicated by a measurement of C-peptide production, and (5) a hemoglobin A1C value less than about 7% The diabetic condition in the subject may be Type I diabetes mellitis or Type II diabetes mellitis. The dosage of the treatment may comprise a dosage which results, for example, in an average daily blood concentration of the flexible heteroarotinoid in a range of from about 0.1 μM to less than 10 μM, an average daily blood concentration of the flexible heteroarotinoid in a range of from about 0.5 μM to about 5 μM, or an average daily blood concentration of the flexible heteroarotinoid in a range of from about 0.5 μM to about 1 μM.

In one embodiment, the presently disclosed inventive concept is a method of treating a condition or disease characterized by cellular accumulation of unfolded or misfolded protein leading to endoplasmic reticulum stress (ERS) in a subject in need of such treatment, comprising, administering to the subject an amount of a flexible heteroarotinoid as defined by at least one of Formulas (I), (II), (Ill), (IV), and (V), wherein the amount of the flexible heteroarotinoid is effective in causing a reduction in cellular ERS in the subject. In particular non-limiting embodiments, the flexible heteroarotinoid may be selected from SHetA2, SHetA3, SHetA4, SHetA19, and SHet[2, and combinations thereof. In the method, the reduction in cellular ERS in the subject may be characterized by a reduction in a biomarker characteristic of ERS. For example, the biomarker may be selected from the group consisting of at least one of HSPA5, GRP94, CHOP, IRE1, HRD1, WFS1, XBP-1, GADD34, GADD153, PDI, ATF3, p58, Fkbpll, Erp 72, ATF4, TRIB3, and Ero1α.

All of the assay methods listed herein are well within the ability of one of ordinary skill in the art given the teachings provided herein.

While the presently disclosed inventive concept(s) is described herein in connection with certain embodiments so that aspects thereof may be more fully understood and appreciated, it is not intended that the presently disclosed inventive concept(s) be limited to these particular embodiments. On the contrary, it is intended that all alternatives, modifications and equivalents are included within the scope of the presently disclosed inventive concept(s) as defined herein. Thus the examples described above, which include preferred embodiments, will serve to illustrate the practice of the presently disclosed inventive concept(s), it being understood that the particulars shown are by way of example and for purposes of illustrative discussion of preferred embodiments of the presently disclosed inventive concept(s) only and are presented in the cause of providing what is believed to be the most useful and readily understood description of procedures as well as of the principles and conceptual aspects of the invention. Changes may be made in the formulation of the various compositions described herein or in the steps or the sequence of steps of the methods described herein without departing from the spirit and scope of the presently disclosed inventive concept(s).

### REFERENCES

The following references, to the extent that they provide exemplary procedural or other details supplementary to those set forth herein, are specifically incorporated herein by reference.
1. Marciniak, S. J. and Ron, D. Endoplasmic reticulum stress signaling in disease. Physiol Rev,86: 1133-1149, 2006.
2. Yoshida, H. ER stress and disease. FEBS J, 274: 630-658, 2007.
3. Zhao, L. and Ackerman, S. Endoplasmic reticulum stress in health and disease. Curr Op Cell Biol, 18: 444-452, 2006.
4. Kane, R C., Bross, P. F., Farrell, A. T., and Pazdur, R Velcade: US FDA approval for the treatment of multiple myeloma progressing on prior therapy. Oncologist, 8: 508-513, 2003.
5. Wright, J. J. Combination therapy of Bortezomib with novel targeted agents: an emerging treatment strategy. Clin Cancer Res, 16: 4094-4104, 2010.
6. Massey, A. J., Williamson, D. S., Browne, H., Murray, I. B., Dokurno, P., Shaw, T., Macias, A T., Daniels, Z., Geoffroy, S., Dopson, M., Lavan, P., Matassova, N., Francis, G. L., Graham, C. J., Parsons, RL Y., Padfield, A, Corner, M., Drysdale, M. I., and Wood, M. A novel, small molecule inhibitor of Hsc70/Hsp70 potentiates Hsp90 inhibitor induced apoptosis in HCT116 colon carcinoma cells. Cancer Chemotherapy & Pharmacology, 66: 535-545, 2010.
7. Guruswamy, S., Lightfoot, S., Gold, M., Hassan, R., Berlin, K. D., Ivey, R T., and Benbrook, D. M. Effects of retinoids on cancerous phenotype and apoptosis in organotypic culture of ovarian carcinoma. I. Nat. Cancer Inst., 93: 516-525, 2001.
8. Benbrook, D. M. Mechanism of SHetA2 in Ovarian Cancer. University of Oklahoma Health Sciences Center, 2009.
9. McCullough, K. D., Martindale, J. L., Klotz, L. O., Aw, T. Y., and Holbrook, N. J. Gadd153 sensitizes cells to endoplasmic reticulum stress by down-regulating BcI2 and perturbing the cellular redox state. Mol Cell Biol, 21: 1249-1259, 2001.
10. Liu, T.-Z., Hannafon, B., Gill, L., Kelly, B., and Benbrook, D. M. Flex-Hets differentially induce apoptosis in cancer over normal cells by directly targeting mitochondria. Mol. Cancer Ther., 6: 1814-1822, 2007.
11. Liu, T., Masamha, P., Chengedza, S., Berlin, K. D., Lightfoot, D., He, F., and Benbrook, D. M. Development of Flexible-Heteroarotinoids (Flex-Hets) for kidney cancer. Molecular Cancer Therapeutics, 8: 1227-1238, 2009.
12. Deng, J., Lu, P. D., Zhang, Y., Scheuner, D., Kaufman, R J., Sonenberg, N., Harding, H. P., and Ron, D. Translational repression omediates activation of Nuclear Factor kappa B by phosphorylated translation initiation factor 2. Mol Cell Biol, 24: 10161-10168, 2004.
13. Chengedza, S. and Benbrook, D. M. NF-κB is involved in SHetA2 circumvention of TNF-a resistance, but not induction of intrinsic apoptosis. Anti-Cancer Drugs, 21: 297- 305, 2010.
14. Yang, Q., Kim, Y. S., Lin, Y., Lewis, J., Neckers, L., and Liu, Z. G. Tumournecrosis factor receptor 1 mediates endoplasmic reticulum stress-induced activation of the MAP kinase JNK. EMBO Rep, 7: 622-627, 2006.
15. Lin, Y., Liu, x., Yue, P., Benbrook, D. M., Berlin, K. D., Khuri, F. R, and S.-Y., S. Involvement of c-FLIPand survivin down-regulation in flexible heteroarotinoid-induced apoptosis and enhancement of TRAIL-initiated apoptosis in lung cancer cells. Molecular Cancer Therapetics, 7: 3556-3565, 2008.
16. Diehl, J. A, Yang, W., Rimerman, R A, Xiao, H., and Emili, A Hsc70 regulates accumulation of cyelin D1 and cyclin D1-dependent protein kinase. Mol Cell Biol, 23: 1764-1774, 2003.
17. Masamha, C. P., Liu, T., Benbrook, D. M. SHetA2 targets cyelin D1 for proteasomal degradation through a GSK3β-independent mechanism leading to G1 cell cycle arrest. In: AACR-NCI-EORTC International Conference on Molecular Targets and Cancer Therapeutics, San Diego, Calif., USA, Oct. 22-26 2007.
18. Shiota, M., Kusakabe, H., Izumi, Y., Hikita, Y., Nakao, T., Funae, Y., Miura, K., and lwaoH. Heat shock cognate protein 70 is essential for Akt signaling in endothelial function. Arteriosc1er Thromb Vase Biol, 30: 491-497, 2010.
19. Myers, T., Chengedza, S., Lightfoot, S., Pan, Y., Dedmond, D., Cole, L., Tang, Y., and Benbrook, D. M. Flexible Heteroarotinoid (Flex-Het) SHetA2 inhibits angiogenesis in vitro and in vivo. Investigational New Drugs, 27: 304-318, 2008.
20. Valente, E. M., Abou-Sleiman, P. M., Caputo, V., Muqit, M. M., Harvey, K., Gispert, S., Ali, Z., Del Turco, D., Bentivoglio, A. R, Healy, D. G., and al, e. Hereditary early-onset Parkinsons disease caused by mutations in P NK1. Science, 304: 1158-1160, 2004.
21. Kelly, W. published in abstract form only.
22. Schneider, H.-C., Berthold, J., Bauer, M. F., Dietmeier, K., Guiard, B., Brunner, M., and Neupert, W. Mitochondrial Hsp70/M1M44 complex facilitates protein import. Nature, 371: 768-774, 1994.
23. Deocaris, C. C., Kaul, S. C., and Wadhwa, R. On the brotherhood of the mitochondrial chaperones mortalin and heat shock protein 60. Cell Stress & Chaperones, 11: 116-128, 2006.
23a. Lu, W.-J., Lee, N.P., Kaui, S.C., Lan, F., Poon, R.T.P., Wadhwa, R., Luk, J. M. Mortalin-p53 interaction in cancer cells is stress dependent and constitutes a selective target for cancer therapy. Cell Death and Differentiation, 18, 1046-1056, 2011.
24. Bence, N. F., Sampat, R. M., and Kopito, R. R. Impairment of the ubiquitin-proteasome system by protein aggregation. Science, 292: 1552-1555, 2001.
25. Schaffar, G., Breuer, P., Boteva, R, Behrends, C., Tzvetkov, N., Strippel, N., Sakahira, H., Siegers, K., Hayer-Hartl, M., and Hart!, F. U. Cellular toxicity of polyglutamine expansion proteins: mechanism of transcription factor deactivation. Mol Cell, 15: 95-105, 2004.
26. Sugars, K. L. R, D. C. Transcriptional abnormalities in Huntington disease. Trends Genet, 19: 233-238, 2003.
27. Ohoka, N., Yoshii, S., Hattori, T., and Onozaki, H. TRB3, a novel ER stress-inducible induced via ATF4-CHOP pathway and cell death. EMBO J 24: 1243-1255, 2005.
28. Vos, M. L, Hageman, L, Carra, S., and Kampinga, H. H. Structural and functional diversities between members of the human HSPB, HSPH, HSPA and DNAJ chaperone families. Biochem, 47: 7001-7011, 2008.
29. Bercovich, B., Stancovski, L,Mayer, A, Blumenfeld, N., Laszlo, A., Schwartz, A L., and Ciechanover, A. Ubiquitin-dependent Degradation of Certain Protein Substrates in Vitro Requires the Molecular Chaperone Hsc70 J Biol Chem, 272: 9002-9010, 1997.
30. Goldfarb, S. B., Kashlan, O. B., Watkins, J. N., Suaud, L., Yan, W., Kleyman, T. R, and Rubenstein, R C. Differential effects of Hsc70 and Hsp70 on the intracellular trafficking and functional expression of epithelial sodium channels. Proceedings of the National Academy of Sciences, 103: 5817-5822, 2006.
31. Chiang, H. L., Terlecky, S. R, Plant, C. P., and Dice, J. F. A role for a 70-kilodalton heat shock protein in lysosomal degradation of intracellular proteins. Science, 246: 382-385, 1989.
32. Plumier, J. C., Ross, B. M., Currie, R. W.; Angelidis, C. E., Kazlaris, H., Kollias, G., and Pagoulatos, G. N. Transgenic mice expressing the human heat shock protein 70 have improved post-ischemic myocardial recovery. J Clin Invest, 95: 1854-1960, 1995.
33. Mestril, R., Giordano, F. J. A.G., and Dillmann, W. H. Adenovirus-mediated gene transfer of a heat shock protein 70 (hsp 70i) protects against simulated ischemia. J Mol Cell Cardiol. ,282351-2358, 1996.
34. Trost, S. U., Omens, J. H., Karlon, W. J., Meyer, M., Mestril, R, and Covell, J. W. Dillmann W H. Protection against myocardial dysfunction after a brief ischemic period in transgenic mice expressing inducible heat shock protein 70. J Clin Invest, 101: 855-862, 1998.
35. Marber, M. S., Mestril, R, Chi, S. H., Sayen, M. R, Yellon, D. M., and Dillmann, W. H. Overexpression of the rat inducible 70-kD heat stress protein in a transgenic mouse increases the resistance of the heart to ischemic injury. J Clin Invest, 95: 1446-1456, 1995.
36. Gerra, G., Calbiani, B., Zaimovic, A., Sartori, R., Ugolotti, G., Ippolito, L., Delsignore, R, Rustichelli, P., and Fontanesi, B. Regional cerebral blood flow and comorbid diagnosis in abstinent opioid addicts. Psychiatry Res., 83: 117-126, 1998.
37. Currie, R W., Karmazyn, M., Kloc, M., and Mailer, K Heat-shock response is associated with enhanced postischemic ventricular recovery. Circ Res, 63: 543-549, 1988.
38. Kim, Y. K., Suarez, J., Hu, Y., McDonough, P. M., Boer, C., Dix, D. J., and Dillmann, W. H. Deletion of the inducible 70-kDa heat shock protein genes in mice impairs cardiac contractile function and calcium handling associated with hypertrophy” Cirulation, 11 3: 2589-2597,2006.
39. Terada, S., Kinjo, M., Aihara, M., Takei, Y., and Hirokawa, N. Kinesin-1/Hsc70- dependent mchanism of slow axonal trnaport and its relation to fast axonal transport. EMBO J, 29: 843-854, 2010.
40. Burbulla, L. F., Schelling, C., Kato, H., Rapaport, D., Woitalla, d., Schiesling, c, Shchulte, c., Sharma, M., IIlig, T., Bauer, P., Jung, S., Nordheim, A., Schols, L., Riess, O., and Kruger, R Dissecting the role of the mitochondrial chaperone mortalin in Parkinson's disease: functional impact of disease-related variants on mitochondrial homeostasis. Hum Mol Gen, 10: 1-16, 2010.
41. Wadhwa, R, Taira, K, and Kaul, S. C. Can mortalin be a candidate target for cancer therapy? Cancer Ther, 1: 173-178, 2003.
42. Wadhwa, R, Pereira-Smith, O. M., Reddel, R R, Sugimoto, Y., Mitsui, Y., and Kaul, S. C. Correlation between Complementation Group for Immortality and the Cellular Distribution of Monalin. Experimental Cell Research, 216: 101-106, 1995.
43. Ran, Q., Wadhwa, R, Kawai, R, Kaul, S. C., Sifers, R. N., Bick, R J., Smith, J. R, and Pereira-Smith, O. M. Extramitochondrial Localization of Mortalin/mthsp70/PBP74/GRP75. Biochemical and Biophysical Research Communications, 275: 174-179, 2000.
44. Webster, T. J., Naylor, D. J., Hartman, D. J., Hoj, P. B., and Hoogenraad, N. J. Cdna Cloning and Efficient Mitochondrial Import of Pre-Mthsp70 from Rat-Liver. DNA and Cell Biology, 13: 1213-1220, 1994.
45. Rehling, P., Brandner, K, and Pfanner, N. Mitochondrial import and the twin-pore translocase. Not Rev Mol Cell Biol, 5: 519-530,2004.
46. Vogel, M., Mayer, M. P., and Bukau, B., Allosteric Regulation of Hsp70 Chaperones Involves a Conserved Interdomain Linker. Journal of Biological Chemistry, Vol. 281, No. 50, pp. 38705-38711, 12/15/2006.
47. Stocki, P., Morris, N. J., Preisinger, C., Wang, X. N., Kolch, W., Multhoff, G., Dickinson, A. M., Identification of potential HLA class I and class II epitope precursors associated with heat shock protein 70 (HSPA). Cell Stress and Chaperones, Vol. 15, pp. 729-741 (2010).
48. Ballabio, A. and Gieselmann, V. Lysosomal disorders: From storage to cellular damage. Biochimica et Biophysica Acta, 1793 (2009) 684-696.
49. Nakatogawa, H., Suzuki, K., Kamada, Y., and Ohsumi, Y. Dynamics and diversity in autophagy mechanisms: lessons from yeast. Nat Rev Cancer, 10: 458-467, 2009.
50. Li, J., Ni, M., Lee, B., Barron, E., Hinton, D. R., and Lee, A. S. The unfolded protein response regulator GRP78/BiP is required for endoplasmic reticulum integrity and stress-induced autophagy in mammalian cells. Cell Death & Differentiation, 15: 1460-1471, 2008.
51. Bennett, H. L., Fleming, J. T., O′Prey, J., Ryan, K. M., and Leung, H. Y. Androgens modulate autophagy and cell death via regulation of the endoplasmic reticulum chaperone glucose-regulated protein 78/BiP in prostate cancer cells. Cell Death & Disease, 1: e72, 2010.
52. Bejarano, E. Cuervo, A. M. Chaperone-Mediated Autophagy. Proc. Am. Thorac. Soc. 7:29-89,20l0.
53. Li, B., et al., Proteomic profiling of differentially expressed proteins from Bax inhibitor-1 knockout and wild type mice. Molecules & Cells, 2012. 34(1): p. 15-23.
54. Johannesen, J., et al., Is mortalin a candidate gene for T1DM ? Autoimmunity, 2004. 37(6-7): p. 423-30.
55. Wadhwa, R., et al., Identification of a novel member of mouse hsp7O family. Its association with cellular mortal phenotype. Journal of Biological Chemistry, 1993. 268(9): p. 6615-6621.
56. Kaul, S. C., et al., Mouse and human chromosomal assignments of mortalin, a novel member of the murine hsp70 family of proteins. FEBS Letters, 1995. 361(2-3): p. 269-272.
57. Oksenberg, J. R. and S.L. Hauser, New insights into the immunogenetics of multiple sclerosis. Current opinion in neurology, 1997. 10(3): p. 181-185.
58. Rioux, J.D., et al., Genomewide Search in Canadian Families with Inflammatory Bowel Disease Reveals Two Novel Susceptibility Loci. American journal of human genetics, 2000. 66(6): p. 1863-1870.
59. Los, H., G. Koppelman, and D. Postma, The importance of genetic influences in asthma. European Respiratory Journal, 1999. 14(5): p. 1210-1227.
60. Li, W., Q. Yang, and Z. Mao, Chaperone-mediated autophagy: machinery, regulation and biological consequences. Cell. Mol. Life Sci, 2011. 68: p. 749-763.
61. Venugopal, B., et al., Chaperone-mediated autophagy is defective in mucolipidosis type IV. Journal of Cellular Physiology, 2009. 219(2): p. 344-353.

What claimed is:

1. A method of treating a diabetic or pre-diabetic condition in a subject in need of such treatment, the method comprising the step of:
administering to the subject a therapeutically-effective amount of a flexible heteroarotinoid selected from the group consisting of SHetA2, SHetA3, SHetA4, SHetA19, and SHetC2, and combinations thereof.

2. The method of claim 1, wherein the treatment results in an increase in insulin sensitivity in the subject.

3. The method of claim 1, wherein the treatment results in a decrease in insulin resistance in the subject.

4. The method of claim 1, wherein the treatment results in a decrease in blood glucose levels in the subject.

5. The method of claim 1, wherein the treatment results in a stabilization or increase in C-peptide production in the subject.

6. The method of claim 1, wherein the treatment results in a stabilization of beta cell mass in the subject as indicated by a measurement of C-peptide production in the subject.

7. The method of claim 1, wherein the treatment results in a hemoglobin A1C value less than about 7% in the subject.

8. The method of claim 1, wherein the diabetic condition is Type II diabetes mellitis.

9. The method of claim 1, wherein the diabetic condition is Type I diabetes mellitis.

10. The method of claim 1, wherein the therapeutically effective amount of the flexible heteroarotinoid comprises a dosage which results in an average daily blood concentration of the flexible heteroarotinoid in a range of from about 0.1 μM to less than 10 μM.

11. The method of claim 10, wherein the dosage results in an average daily blood concentration of the flexible heteroarotinoid in a range of from about 0.5 μM to about 5 μM.

12. The method of claim 10, wherein the dosage results in an average daily blood concentration of the flexible heteroarotinoid in a range of from about 0.5 μM to about 1 μM.

13. A method of treating a condition or disease characterized by cellular accumulation of unfolded or misfolded protein leading to endoplasmic reticulum stress (ERS) in a subject in need of such treatment, the method comprising the step of:
administering to the subject an amount of a flexible heteroarotinoid, wherein the amount of the flexible heteroarotinoid is effective in causing a reduction in cellular ERS in the subject, and wherein the flexible heteroarotinoid is selected from the group consisting of SHetA2, SHetA3, SHetA4, SHetA19, and SHetC2, and combinations thereof.

14. The method of claim 13, wherein the reduction in cellular ERS in the subject is characterized by a reduction in a biomarker characteristic of ERS.

15. The method of claim 14, wherein the biomarker is selected from the group consisting of at least one of HSPA5, GRP94, CHOP, IRE1, HRD1, WFS1, XBP-1, GADD34, GADD153, PDI, ATF3, p58, Fkbp11, Erp 72, ATF4, TRIB3, and Ero1α.

16. A method of treating a condition or disease characterized by cellular accumulation of unfolded or misfolded protein leading to endoplasmic reticulum stress (ERS) in a subject in need of such treatment, the method comprising the step of:
administering to the subject an amount of a flexible heteroarotinoid, wherein the amount of the flexible heteroarotinoid is effective in causing a reduction in cellular ERS in the subject, and wherein the flexible heteroarotinoid is defined by at least one of Formulas (I) and (II): 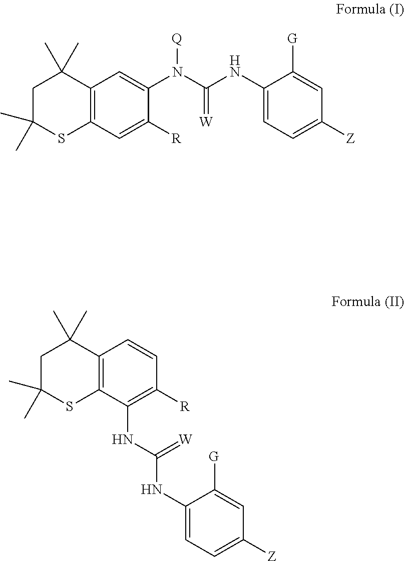
wherein R denotes H, CH₃, or OCH₃;
Q denotes H or i-C₃H₇;
W denotes O or 5;
G denotes H or CH₃; and
Z denotes NO₂, CO₂Et, CO₂-n- C₄H₉, or SO₂NH₂.

17. The method of claim 16, wherein the reduction in cellular ERS in the subject is characterized by a reduction in a biomarker characteristic of ERS.

18. The method of claim 17, wherein the biomarker is selected from the group consisting of at least one of HSPA5, GRP94, CHOP, IRE1, HRD1, WFS1, XBP-1, GADD34, GADD153, PDI, ATF3, p58, Fkbp11, Erp 72, ATF4, TRIB3, and Ero1α.
